(19) United States
(12) Patent Application Publication
(10) Pub. No.: US 2008/0311574 A1
(43) Pub. Date: Dec. 18, 2008

(54) Novel Missense Mutations and Single Nucleotide Polymorphisms in the Rabphillin-3A-Like Gene and Uses Thereof

(76) Inventors: Upender Manne, Birmingham, AL (US); Venkat Rao Katkoori, Vestavia Hills, AL (US); Chakrapani Chatla, Seconderabad (IN)

Correspondence Address: Ballard Spahr Andrews & Ingersoll, LLP, SUITE 1000, 999 PEACHTREE STREET, ATLANTA, GA, 30309-3915, US

(21) Appl. No.: 11/908,342

(22) PCT Filed: Mar. 13, 2006
(86) PCT No.: PCT/US2006/008830

§ 371 Date: Jun. 16, 2008

Related U.S. Application Data
(60) Provisional application No. 60/660,938, filed on Mar. 11, 2005.

(51) Int. Cl. C12Q 1/68; G06F 17/30; G06F 7/00

(52) U.S. Cl. 435/6; 707/104.1; 707/6; 707/103 R; 707/E17.055; 707/E17.044; 707/E17.014

(57) ABSTRACT

The invention relates to methods and compositions of matter for determining or predicting aggressiveness of a subject's tumor, for determining a subject's predisposition to cancer, for diagnosing cancer in a subject, and for selecting a therapy for a subject with cancer. Also provided are methods and compositions of matter for determining a Rabphillin-3A-Like gene genotype in a subject and for characterizing a Rabphillin-3A-Like gene in a subject.

### CROSS-REFERENCE TO RELATED APPLICATIONS

This application claims benefit of U.S. Provisional Application No. 60/660,938, filed Mar. 11, 2005, which application is hereby incorporated herein by reference in its entirety.

This invention was made with government support under grant RO1-CA98932-01 from the National Cancer Institute, National Institute of Health. The government has certain rights in the invention.

### FIELD OF THE INVENTION

The present invention relates generally to the treatment, diagnosis, and prevention of cancer and to the clinical implications of novel missense mutations and single nucleotide polymorphisms in the Rabphillin-3A-like gene.

### BACKGROUND OF THE INVENTION

In 2006, colorectal adenocarcinoma (CRC) will be diagnosed in approximately 148,610 Americans and will be responsible for 55,170 deaths [American Cancer Society. Cancer facts and figures. 2006. Atlanta, Ga.]. Currently, treatment decisions are based on clinical and pathologic staging of CRC; however, the tumor stage alone may not be the best indicator, since groups of patients with tumors of identical stage have different treatment responses and outcomes. Therefore, several investigations have focused on identifying molecular marker(s), including single mutations in specific genes that are associated with the aggressiveness of CRCs. Such molecular changes need to be well characterized before conducting large population-based epidemiological studies in order to focus on appropriate candidates needed to identify high risk groups of patients with aggressive tumors, in designing treatment strategies and in predicting prognosis.

The majority of CRCs are sporadic, whereas less than 5% of all tumors comprise familial types. Most CRCs have been hypothesized to arise from adenomas [Muto, T., H. J. Bussey, and B. C. Morson, The evolution of cancer of the colon and rectum. Cancer, 1975. 36(6): p. 2251-70] through the accumulation of several genetic alterations that dysregulate cell growth [Vogelstein, B., et al., Genetic alterations during colorectal-tumor development. New England Journal of Medicine, 1988. 319(9): p. 525-32; Fearon, E. R., et al., Identification of a chromosome 18q gene that is altered in colorectal cancers. Science, 1990. 247(4938): p. 49-56.; Hamilton, S. R., The molecular genetics of colorectal neoplasia. Gastroenterology, 1993. 105(1): p. 3-7.]). Tumor suppressor genes have been implicated in the development of a wide variety of human malignancies including CRC, and have been shown to be related to chromosomal rearrangements, particularly mutations and/or deletions [Kern, S. E., et al., Oncogenic forms of p53 inhibit p53-regulated gene expression. Science, 1992. 256(5058): p. 827-30.; Vogelstein, B., et al., Genetic alterations during colorectal-tumor development. New England Journal of Medicine, 1988. 319(9): p. 525-32.; Yamaguchi, A., et al., Expression of p53 protein in colorectal cancer and its relationship to short-term prognosis. Cancer, 1992. 70(12): p. 2778-84; Kern, S. E., et al., Clinical and pathological associations with allelic loss in colorectal carcinoma [published erratum appears in JAMA 1989 Oct. 13; 262(14):1952]. Journal of the American Medical Association, 1989. 261(21): p. 3099-103.].

Loss of heterozygosity (LOH), defined as a loss of one allele at a constitutional (germline) heterozygous locus, has been accepted as a hallmark of one of the two hits required for the inactivation of tumor suppressor genes in cancer. The LOH on chromosome 17p is one of the common genetic aberrations in many tumors. Genetic regions of LOH on chromosome 17p are frequently reported in CRCs [Kern, S. E., et al., Oncogenic forms of p53 inhibit p53-regulated gene expression. Science, 1992. 256(5058): p. 827-30.; Freedman, A. N., et al., Familial and nutritional risk factors for p53 overexpression in colorectal cancer. Cancer Epidemiology, Biomarkers & Prevention, 1996. 5(4): p. 285-91.; Delattre, O., et al., Multiple genetic alterations in distal and proximal colorectal cancer. Lancet, 1989. 2(8659): p. 353-6.; Boland, C. R., et al., Microallelotyping defines the sequence and tempo of allelic losses at tumour suppressor gene loci during colorectal cancer progression. Nature Medicine, 1995. 1(9): p. 902-9.]. Boland et al. [Boland, C. R., et al., Microallelotyping defines the sequence and tempo of allelic losses at tumour suppressor gene loci during colorectal cancer progression. Nature Medicine, 1995. 1(9): p. 902-9.] demonstrated a high proportion of ˜70% LOH at chromosome 17p in CRCs in one such study. Such genetic alterations are a hall mark for the presence of tumor suppressor genes and suggest for the existence of additional tumor suppressor genes besides p53, which is known to occur at this region [McDonald, J. D., et al., Physical mapping of chromosome 17p13.3 in the region of a putative tumor suppressor gene important in medulloblastoma. Genomics, 1994. 23(1): p. 229-32.; Biegel, J. A., et al., Evidence for a 17p tumor related locus distinct from p53 in pediatric primitive neuroectodermal tumors. Cancer Res, 1992. 52(12): p. 3391-5.; Vogelstein, B., et al., Allelotype of colorectal carcinomas. Science, 1989. 244(4901): p. 207-11.; Vogelstein, B., et al., Genetic alterations during colorectal-tumor development. New England Journal of Medicine, 1988. 319(9): p. 525-32.; Fearon, E. R. and B. Vogelstein, A genetic model for colorectal tumorigenesis. Cell, 1990. 61(5): p. 759-67.; Thiagalingam, S., et al., Mechanisms underlying loss of heterozygosity in human colorectal cancers. Proc Natl Acad Sci USA, 2001. 98(5): p. 2698-702.].

A study by Smith et al. in 1999 [Smith, J. S., Tachibana, I., Allen, C., Chiappa, S. A., Lee, H. K., McIver, B., Jenkins, R. B., and Raffel, C. Cloning of a human ortholog (RPH3AL) of (RNO)Rph3a1 from a candidate 17p13.3 medulloblastoma tumor suppressor locus. Genomics, 59: 97-101, 1999] identified a candidate tumor suppressor gene, a human ortholog of the Rabphillin-3A-like gene (RPH3AL) (GenBank # AF129812) at the 17p13.3 locus. Simultaneously, RPH3AL was cloned and sequenced in medullobastoma tumors [Smith, J. S., et al., Cloning of a human ortholog (RPH3AL) of (RNO)Rph3a1 from a candidate 17p13.3 medulloblastoma tumor suppressor locus. Genomics, 1999. 59(1): p. 97-101.]. Recently, a study of CRC from Europe reported 6 missense point mutations in the coding region of the RPH3AL gene and suggested that RPH3AL may play a tumor suppressor role in a small proportion (12%) of CRCs [Goi, T., et al., Mutations of rabphillin-3A-like gene in colorectal cancers. Oncol Rep, 2002. 9(6): p. 1189-92].

The full-length coding sequence of RPH3AL and its gene product (315 amino acid residues) have demonstrated considerable homology (77% identity at the amino acid level) with the rat Rph3a1 gene (originally termed Noc2) [Smith, J. S., et al., Cloning of a human ortholog (RPH3AL) of (RNO)Rph3a1 from a candidate 17p13.3 medulloblastoma tumor suppressor locus. Genomics, 1999. 59(1): p. 97-101.]. Although, the precise functions of RPH3AL is not known, the Noc2 gene is known to be involved in the regulation of endocrine exocytosis through its interactions with the cytoskeleton [Kato, M., et al., Physical and functional interaction of rabphilin-3A with alpha-actinin. J Biol Chem, 1996. 271(50): p. 31775-8.; Kotake, K., et al., Noc2, a putative zinc finger protein involved in exocytosis in endocrine cells. J Biol Chem, 1997. 272(47): p. 29407-10] and it has been suggested that the RPH3AL gene product might have an important functional role in a variety of humans cells. Needed in the art, however, are specific genetic correlates in RPH3AL that can be used for prognosis, prediction, diagnosis, and therapy selection.

### SUMMARY OF THE INVENTION

In accordance with the purpose of this invention, as embodied and broadly described herein, this invention relates to treatment, diagnosis, and prevention of cancer based on novel missense mutations, loss of heterozygosity, and single nucleotide polymorphisms in the Rabphillin-3A-Like gene and related genotypes. Thus provided herein are methods and compositions of matter for determining or predicting aggressiveness of a subject's tumor, for determining a subject's predisposition to cancer, for diagnosing cancer in a subject, and for selecting a therapy for a subject with cancer. Also provided are methods and compositions of matter for determining a Rabphillin-3A-Like gene genotype in a subject and for characterizing a Rabphillin-3A-Like gene in a subject.

### BRIEF DESCRIPTION OF THE FIGURES

FIG. 1 shows the DNA sequences obtained from a tumor sample and its matching benign epithelium demonstrate similar nucleotide sequence suggesting that nucleotide change (from wild-type) is a genetic polymorphism.

FIG. 2 shows (A) PCR-CTPP for the UTR-25 (C>A) variant of RPH3AL. Primers are shown with the horizontal arrows below the scheme representing exon-intron structure. The nucleotide at the 3′ end is the cytosine of the F2 primer and the thymine of the R1 primer. The position of the 5′UTR-25 (C>A) variation is indicated by a vertical arrow. (B) The agarose gel (2%) electrophoresis panel represents the PCR amplification of the samples with UTR-25 C/C, A/A and C/A genotypes using the primers indicated below. The size of each PCR product (band) is 209 bps for F1/R2 primers set, 146 bps for F2/R2 primer set and 111 bps for F1/R1 primer set. Lanes 1, 4 & 7 are DNA from a representative case with C/C genotype, lanes 2, 5 & 8 are DNA from a representative case with A/A genotype, and lanes 3, 6 & 8 are DNA from a representative case with C/A genotype. (C) Sequencing analysis of the PCR product with the F1 and R primers.

FIG. 3 shows the Kaplan-Meier analyses to predict the disease recurrence (FIG. 3A) and survival (FIG. 3B) of CRC patients based on the RPH3AL genotypes. These analyses demonstrated that patients homozygous for the A allele or C allele at 5′UTR-25 had higher risk of recurrence within 5 years after surgery compared to patients who were heterozygous for the C/A alleles (log-rank P<0.0001 and P=0.008, respectively) (FIG. 3A). There were significant over-all survival differences between patients with different genotypes: patients with A/A versus C/A; log-rank, P<0.0001; patients with C/C versus C/A; log-rank, P=0.0014; and patients with A/A versus C/C; log-rank, P=0.210 (FIG. 3B).

FIG. 4 shows the Kaplan-Meier survival analyses of Caucasian and African-Americans with CRCs based on p53 ac and the anatomic location of the tumor. (A) Caucasians with proximal CRCs, (B) Caucasians with distal CRCs, (C) African-Americans with proximal CRCs, and (D) African-Americans with distal CRCs.

FIG. 5 shows the Kaplan-Meier survival analyses of Caucasian patients with proximal tumors exhibiting mutations in L3 domain of p53 versus wtp53 gene.

FIG. 6 shows the relationship between the empirical bayes (EB) and the ordinary estimate (OE) of the standardized difference. The x and y axes represent the OE and the EB values respectively.

FIG. 7 shows the mRNA levels of the RPH3AL gene quantified by Real-Time PCR in normal tissue and tumor tissue samples. FIG. 7A shows the mean numbers of RPH3AL mRNA copies, in relation to β-Actin mRNA copies, in tumors (n=28), (0.435±0.078) and in their matching normal tissue samples (1.908±0.601). The differences in mRNA levels were significantly different from each groups of samples (χ², p<0.0001). The RPH3AL mRNA expression was significantly reduced in tumors compared to their matching benign normal colonic epithelial tissues, and the difference between these two groups of tissues was significantly different (χ², p<0.0001) (FIG. 7A). FIG. 7B shows mRNA levels quantified in tumors and in their matching normal tissues based on three genotypes of SNP at 5′UTR-25 of the RPH3AL gene. Analysis of RPH3AL mRNA levels based on three genotypes of SNP at 5′UTR-25 of the RPH3AL gene suggested that there was a significant difference between the tumors and in their matching normal tissues, specifically in the genotypes A/A and C/C, but not in C/A (FIG. 7B).

### DETAILED DESCRIPTION OF THE INVENTION

The present invention may be understood more readily by reference to the following detailed description of preferred embodiments of the invention and the Examples included therein and to the Figures and the Tables and their previous and following description.

Before the present compounds, compositions, articles, devices, and/or methods are disclosed and described, it is to be understood that this invention is not limited to specific nucleic acids or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a nucleic acid” includes mixtures of nucleic acids, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. The term “subject” includes domesticated animals, such as cats, dogs, etc., livestock (e.g., cattle, horses, pigs, sheep, goats, etc.), and laboratory animals (e.g., mouse, rabbit, rat, guinea pig, etc.).

The present invention relates to methods for determining or predicting aggressiveness of a subject's tumor, for determining a subject's predisposition to cancer, for diagnosing cancer in a subject, and for selecting a therapy for a subject based on the subject's Rabphillin-3A-Like gene genotype. A specific site in the 5′ UTR region of the Rabphillin 3A-like gene sequence is polymorphic, i.e., the nucleotide at a specific position or at specific positions varies across a population of subjects such that the nucleotide can be a C or an A or a subset thereof at the specific position.

Therefore, as utilized herein, the term “polymorphic” or “polymorphic site” means that at one or more specific positions in the 5′ UTR region of the Rabphillin 3A-like gene nucleotide sequence, the most commonly found nucleotide or another nucleotide that differs from the most commonly found nucleotide can be identified at the specific site across a population of subjects. Therefore, the term “polymorphic” or “polymorphism” encompasses both the most commonly found nucleotide(s) and another nucleotide(s) found at a specific site(s). For example, position −25 of the 5′ UTR region of the Rabphillin 3A-like gene sequence is polymorphic, wherein the most commonly found nucleotide at position −25 of the 5′ UTR region of the Rabphillin 3A-like gene is C and another nucleotide found at this polymorphic site is A. Therefore, when one of skill in the art is analyzing this site, they can determine which of the two nucleotides (C or A) is present at this site. “Polymorphism” also includes combinations of polymorphisms at more than one position in the 5′ UTR region of the Rabphillin 3A-like gene. Polymorphisms may provide functional differences in the genetic sequence, through changes in the encoded polypeptide, changes in mRNA stability, binding of transcriptional and translation factors to the DNA or RNA, and the like. The polymorphisms are also used as single nucleotide polymorphisms (SNPs) to detect genetic linkage to phenotypic variation in activity and expression of the Rabphillin 3A-like gene.

These methods of the invention are relevant to various types of tumors and various types of cancers including, for example, colorectal cancer, lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumours, myelomas, AIDS-related lymphomas or sarcomas, metastatic cancers, transitional cell carcinoma of ureter and bladder, bladder cancer, nervous system cancer, squamous cell carcinoma of head and neck, neuroblastoma/glioblastoma, astrocytoma, brain cancer, ovarian cancer, basal cell carcinoma, skin cancer, biliary carcinoma, cholangeo carcinoma, angiosarcoma, liver cancer, melanoma, squamous cell carcinomas of the mouth, throat, larynx, tongue, anus, bladder, and lung, small cell carcinoma of lung, large cell carcinoma of lung, adenocarcinoma of lung, rhabdomyosarcoma, gastrointestinal stronal tumors, gastric cancer, adenocarcinomas of stomach, intestinal cancer, colon cancer, rectal cancer, cervical cancer, cervical carcinoma, breast cancer, epithelial cancer, follicular carcinoma of thyroid, medullary carcinoma of thyroid, papillary carcinoma of thyroid, retinoblastoma, phaeochromocytoma, renal cancer, bone cancer, chondrosarcoma, osteosarcoma, ewings sarcoma, seminoma, teratoma, dermoid tumors, embryonal cell carcinoma, choriocarcinoma, endometrial cancer, uterine cancer, vaginal cancer, squamous cell carcinoma of penis, genitourinary cancer, pulmonary cancer, esophageal carcinoma, head and neck carcinoma, hematopoietic cancers, testicular cancer, colo-rectal cancers, prostatic cancer, or pancreatic cancer.

Provided herein is a method for determining or predicting aggressiveness of a subject's tumor, comprising comparing the subject's Rabphillin-3A-Like gene genotype with one or more reference genotypes, wherein the reference genotype or genotypes correlate with aggressive tumor growth, a similar genotype in the subject's Rabphillin-3A-Like gene as compared to the reference genotype or genotypes indicating an aggressive tumor in the subject. Specifically, provided herein is a method for determining or predicting aggressiveness of a subject's tumor, comprising comparing the subject's Rabphillin-3A-Like gene genotype with one or more reference genotypes. When the reference genotype or genotypes correlates with aggressive tumor growth, a similar genotype in the subject's Rabphillin-3A-Like gene as compared to the reference genotype or genotypes indicates an aggressive tumor in the subject. Alternatively, when the reference genotype correlates with non-aggressive tumor growth and the genotype in the subject's Rabphillin-3A-Like gene as is dissimilar as compared to the reference genotype or genotypes, an aggressive tumor in the subject is present or indicated.

As used herein, “determining” aggressiveness refers to assessing the present state of invasiveness or rapid increase in tumor size. By “predicting” is meant assessing the likelihood of rapid growth or invasiveness of a tumor. The tumor can be a solid tumor and can be a malignant tumor, and more specifically can be a colorectal tumor. Some genotypes in certain ethnicities can also be of further assistance in determining the aggressiveness of a tumor. For example, a non-hispanic white colorectal cancer patient that has the genotype A/A, has a higher likelihood of having an aggressive tumor.

The reference genotype as used in the methods herein comprises, for example, the nucleotides at untranslated regions of the gene, including, for example, position −25 of the 5′ untranslated regions of exon 2 of the Rabphillin-3A-Like gene. An example of a reference genotype that correlates with tumor aggressiveness, predisposition to cancer, diagnosis of cancer is A/A. Examples of reference genotypes that correlate with non-aggressiveness, absence of a predisposition to cancer or absence of cancer are C/A. The reference genotype can further comprise other nucleotides in untranslated and translated regions of the gene.

Also provided, is a method for determining or predicting aggressiveness of a subject's tumor as described herein and further comprising comparing other nucleotides within the genomic region of the subject's Rabphillin-3A-Like gene genotype with nucleotides in one or more reference genotypes, wherein the reference genotype or genotypes correlate with aggressive tumor growth. A similar nucleotide within the genomic region of the subject's Rabphillin-3A-Like gene as compared to the reference genotype or genotypes indicates an aggressive tumor in the subject. Other nucleotides within the genomic region of the subject's Rabphillin-3A-Like gene genotype can include SNPs. Example of SNPs within the genomic region of the subject's Rabphillin-3A-Like gene include but are not limited to SNP clusted IDs rs4985611, rs7215343, rs7223403, rs9891032, rs9907777, rs9915104, rs11356209, rs11356210, rs11383870, rs11650641, rs12942009, rs12942039, and rs12949751.

Also, provided herein is a method for determining a subject's predisposition to cancer, comprising comparing the subject's Rabphillin-3A-Like gene genotype with one or more reference genotypes, wherein the reference genotype or genotypes correlate with a predisposition to cancer, a similar genotype in the subject's Rabphillin-3A-Like gene as compared to the reference genotype or genotypes indicating the subject's predisposition to cancer. Alternatively, instead of a reference genotype or genotypes that correlate with a predisposition to cancer, a reference genotype that correlates with the absence of such a predisposition can be used, wherein a dissimilar genotype in the subject's Rabphillin-3A-Like gene as compared to the reference genotype or genotypes indicates the subject's predisposition to cancer. The predisposition is, for example, related to colorectal cancer.

Also provided is a method for determining a subject's predisposition to cancer, described herein and further comprising comparing other nucleotides within the genomic region of the subject's Rabphillin-3A-Like gene genotype with nucleotides in one or more reference genotypes, wherein the reference genotype or genotypes correlate with aggressive tumor growth. A similar nucleotide within the genomic region of the subject's Rabphillin-3A-Like gene as compared to the reference genotype or genotypes indicates the subject's predisposition to cancer. Other nucleotides within the genomic region of the subject's Rabphillin-3A-Like gene genotype can include SNPs. Examples of SNPs within the genomic region of the subject's Rabphillin-3A-Like gene include SNPs as described above.

Also provided herein is a method of diagnosing cancer in a subject, comprising, comparing the subject's Rabphillin-3A-Like gene genotype with one or more reference genotypes, wherein the reference genotype or genotypes correlate with cancer, a similar genotype in the subject's Rabphillin-3A-Like gene as compared to the reference genotype or genotypes indicating the subject's cancer diagnosis.

Also provided herein is a method of diagnosing cancer in a subject as described herein and further comprising comparing other nucleotides within the genomic region of the subject's Rabphillin-3A-Like gene genotype with nucleotides in one or more reference genotypes, wherein the reference genotype or genotypes correlate with aggressive tumor growth. A similar nucleotide within the genomic region of the subject's Rabphillin-3A-Like gene as compared to the reference genotype or genotypes indicates the subject's cancer diagnosis. Other nucleotides within the genomic region of the subject's Rabphillin-3A-Like gene genotype can include SNPs. Examples of SNPs within the genomic region of the subject's Rabphillin-3A-Like gene include SNPs as described above.

The invention provides a method of selecting a therapy for a subject with cancer, comprising the steps of comparing the subject's Rabphillin-3A-Like gene genotype with one or more reference Rabphillin-3A-Like gene genotypes, wherein each reference Rabphillin-3A-Like gene genotype is assigned a preferred therapy; and selecting the reference Rabphillin-3A-Like gene genotype most similar to the subject's Rabphillin-3A-Like gene genotype. The preferred therapy for the most similar reference genotype is the selected therapy for the subject. Thus, for example, if the subject has a genotype that correlates with an aggressive form of cancer then it will be similar to a reference genotype associated with the aggressive treatment. Treatment associated with a reference genotype includes various chemotherapeutics, monoclonal antibody therapy, radiation therapy, surgery, or any combination thereof.

Chemotherapeutics include for example, Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other chemotherapeutics include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgraniostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; onnaplatin; osaterone; oxaliplatin; oxaunomycin; paclitaxel analogues; paclitaxel derivatives; palauamine; pahnitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasonie inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfiosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer supplementary potentiating agents can also be selected, which include, for example, tricyclic anti-depressant drugs (e.g., imipramine, desipramine, amitryptyline, clomiprainine, trimipramine, doxepin, nortriptyline, protriptyline, amoxapine and maprotiline); non-tricyclic anti-depressant drugs (e.g., sertraline, trazodone and citalopram); Ca.sup.++ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. The compounds of the invention also can be administered with cytokines such as granulocyte colony stimulating factor.

### Rabphillin 3A-Like Nucleic Acids

Reference sequences of the human Rabphillin 3A-like gene sequence comprising a most commonly found allele are provided herein. Further provided are nucleic acids of the full length human Rabphillin 3A-like gene with one or more point mutations as shown, for example, in Table 1. Also provided herein are fragments of the full length human Rabphillin 3A-like gene, wherein the fragment comprises one or more point mutations, including, for example, one or more of the mutations shown in Table 1.

As utilized herein, “reference sequence” refers to a Rabphillin 3A-like gene sequence or fragment thereof comprising a specific nucleotide at a particular position(s) in the Rabphillin 3A-like gene sequence. Optionally, the reference is the most commonly found nucleotide or allele at the particular position or positions. This reference sequence can be a full-length Rabphillin 3A-like gene sequence or fragments thereof. An example of a full length human Rabphillin 3A-like gene sequence is provided herein as SEQ ID NO: 1. References to nucleotide positions as used throughout correspond to positions of the full length Rabphillin 3A-like gene. Thus, for example, position 262 (C) in SEQ ID NO:1 corresponds to position −25 of the 5′ UTR of the Rabphillin 3A-like gene, position 431-433 of SEQ ID NO:1 corresponds to the nucleotide sequence CCG (codon 49) of the Rabphillin 3A-like gene; position 470-472 of SEQ ID NO:1 corresponds to the nucleotide sequence GCA (codon 62) of the Rabphillin 3A-like gene; position 1193-1195 of SEQ ID NO:1 corresponds to the nucleotide sequence GCT (codon 303) of the Rabphillin 3A-like gene; position 485-487 of SEQ ID NO:1 corresponds to the nucleotide sequence GTC (codon 67) of the Rabphillin 3A-like gene; position 578-580 of SEQ ID NO:1 corresponds to the nucleotide sequence TGC (codon 98) of the Rabphillin 3A-like gene; position 809-811 of SEQ ID NO:1 corresponds to the nucleotide sequence CCC (codon 175) of the Rabphillin 3A-like gene; position 1088-1090 of SEQ ID NO:1 corresponds to the nucleotide sequence GCC (codon 268) of the Rabphillin 3A-like gene; position 1154-1156 of SEQ ID NO:1 corresponds to the nucleotide sequence AGG (codon 290) of the Rabphillin 3A-Like gene

Alternatively, one of skill in the art can utilize a reference sequence or a fragment thereof comprising a nucleotide or allele that is not the most commonly found nucleotide or allele at a specific nucleotide position(s) in the Rabphillin 3A-like gene sequence or can utilize a reference sequence that comprises alternative nucleotides at a specific position. An example of a full length cDNA sequence of the Rabphillin 3A-like gene that comprises such alternative nucleotides at positions (−25 of the 5′ UTR) of the Rabphillin 3A-like gene, (codon 49), (codon 62), (codon 303), (codon 67), (codon 98), (codon 175), (codon 268), and (codon 290) are provided herein as SEQ ID NOs: 2-10, respectively. Therefore, when utilizing this reference sequence or a fragment thereof, the nucleotide at position 262 (−25 of the 5′ UTR) can be C or A; the nucleotide at position 433 (codon 49) can be G or T; the nucleotide at position 472 (codon 62) can be A or C; the nucleotide at position 1194 (codon 303) can be C or T; the nucleotide at position 485 (codon 67) can be G or A; the nucleotide at position 580 (codon 98) can be C or T; the nucleotide at position 809 (codon 175) can be C or T; the nucleotide at position 1089 (codon 268) can be G or C; and the nucleotide at position 1155 (codon 290) can be G or A. respectively.

For example, the present invention provides a reference sequence comprising the nucleotide at position −25 of the 5′ UTR of the Rabphillin 3A-like gene sequence. One of skill in the art can compare this reference sequence to a test sequence and determine if the most commonly found nucleotide (C) is present at position −25 of the 5′ UTR of the test sequence or if another nucleotide (A) is present at position (−25 of the 5′ UTR) of the test sequence. Alternatively, one of skill in the art could compare the test sequence to another reference sequence comprising the nucleotide (A) at position −25 of the 5′ UTR and could determine whether the test sequence has a “C” or an “A” at position −25 of the 5′ UTR.

The reference genotype can comprise nucleotides at other positions, including, for example, nucleotides corresponding to position 431-433 (codon 49), position 470-472 (codon 62), position 1193-1195 (codon 303), or any combination thereof. The reference genotype can comprise a “G” at position (codon 49), which is the most commonly found nucleotide at this position, or a “T” at position (codon 49). Therefore, one of skill in the art can compare this reference sequence to a test sequence and determine if the most commonly found nucleotide (G) is present at position (codon 49) or another nucleotide (T) is present at position (codon 49) of the test sequence. Similarly, the reference sequence can have an “A” corresponding to position 472 (codon 62), which is the most commonly found nucleotide at this position. One of skill in the art can compare this reference sequence to a test sequence and determine if the most commonly found nucleotide (A) is present at position (codon 62) or another nucleotide (C) is present at position (codon 62) of the test sequence. Finally, the reference sequence can have an “C” corresponding to position 1194 (codon 303), which is the most commonly found nucleotide at this position. One of skill in the art can compare this reference sequence to a test sequence and determine if the most commonly found nucleotide (C) is present at position 1194 (codon 303) or another nucleotide (T) is present at position (codon 303) of the test sequence

Thus, provided herein are nucleic acid sequences and fragments comprising one or more of these mutations in the Rabphillin 3A-like gene. Table 1 indicates various mutations and wild type nucleotides at specific points. As used, herein, the term “wild-type” may also be used to refer to the reference sequence comprising the most commonly found allele. It will be understood by one of skill in the art that the designation as “wild-type” is merely a convenient label for a common allele and should not be construed as conferring any particular property on that form of the sequence.

Nucleic acids of interest comprising the polymorphisms provided herein can be utilized as probes or primers. Also provided herein are nucleic acids comprising

(SEQ ID NO: 11)

| | |
|---|---|
| 5′-GAGGGCACAGAGAA CCTGTC-3′ | (F1 primer); |

(SEQ ID NO: 12)

| | |
|---|---|
| 5′-GGAGCACCCGGCTGGGGGTT-3′ | (R1 primer), |

(SEQ ID NO: 13)

| | |
|---|---|
| 5′-CATCTCAGATGTGACTCCCC-3′ | (F21 primer), |

(SEQ ID NO: 14)

| | |
|---|---|
| 5′-GGCCCCAGAGGTACTCACTT-3′ | (R2 primer). |

The complementary sequences of the nucleic acid sequences provided herein are also provided by the present invention. For the most part, the nucleic acid fragments will be of at least about 15 nt, usually at least about 20 nt, often at least about 50 nt. Such fragments are useful as primers for PCR, hybridization screening, etc. Larger nucleic acid fragments, for example, greater than about 100 nt are useful for production of promoter fragments, motifs, etc. For use in amplification reactions, such as PCR, a pair of primers will be used. The exact composition of primer sequences is not critical to the invention, but for most applications the primers will hybridize to the subject sequence under stringent conditions, as known in the art.

By “hybridizing under stringent conditions” or “hybridizing under highly stringent conditions” is meant that the hybridizing portion of the hybridizing nucleic acid, typically comprising at least 15 (e.g., 20, 25, 30, or 50 nucleotides), hybridizes to all or a portion of the provided nucleotide sequence under stringent conditions. The term “hybridization” typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize. Generally, the hybridizing portion of the hybridizing nucleic acid is at least 80%, for example, at least 90%, 95%, or 98%, identical to the sequence of or a portion of the Rabphillin-3A-like gene nucleic acid of the invention, or its complement. Hybridizing nucleic acids of the invention can be used, for example, as a cloning probe, a primer (e.g., for PCR), a diagnostic probe, or an antisense probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Assuming that a 1% mismatch results in a 1° C. decrease in the Tm, the temperature of the final wash in the hybridization reaction is reduced accordingly (for example, if sequence having >95% identity with the probe are sought, the final wash temperature is decreased by 5° C.). In practice, the change in Tm can be between 0.5° C. and 1.5° C. per 1% mismatch. Stringent conditions involve hybridizing at 68° C. in 5×SSC/5×Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and the target nucleic acid. Additional guidance regarding such conditions is readily available in the art, for example, in Sambrook et al., 1989, Molecular Cloning, A Laboratory Manual, Cold Spring Harbor Press, NY; and Ausubel et al. (eds.), 1995, Current Protocols in Molecular Biology, (John Wiley & Sons, NY) at Unit 2.10.

The nucleic acids of the present invention can also be utilized in an array. An array may include all or a subset of the polymorphic sequences listed in Table 9. Usually, such an array will include at least 2 different sequences. The oligonucleotide sequence on the array will usually be at least about 12 nt in length, may be the length of the provided polymorphic sequences, or may extend into the flanking regions to generate fragments of 100 to 200 nt in length. For examples of arrays, see Ramsay (1998) Nat. Biotech. 16:4044; Hacia et al. (1996) Nature Genetics 14:441-447; Lockhart et al. (1996) Nature Biotechnol. 14:1675-1680; and De Risi et al. (1996) Nature Genetics 14:457-460, which are incorporated by reference in their entirety for the methods of making and using arrays.

Nucleic acids may be naturally occurring, e.g. DNA or RNA, and may be double stranded or single stranded. Synthetic analogs of the nucleic acids are also provided. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3″-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without compromising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Further provided is a kit for determining a Rabphillin-3A-Like gene genotype in a subject, comprising one or more amplification primers selected from the group consisting of SEQ ID NOS:11-16; and instructions for determining the subject's genotype using one or more of the primers.

### Identification Methods

Provided herein is a method for determining a Rabphillin-3A-Like gene genotype in a subject comprising identifying nucleotides present in both copies in the subject's Rabphillin-3A-Like gene at a polymorphic site in the 5′ untranslated region of exon 2 of the Rabphillin-3A-Like gene. The identifying step comprises, for example, identifying the nucleotide at position −25 of the 5′ untranslated region of the exon 2 of the Rabphillin-3A-Like gene (Smith et al. 1999). The genotype can be C/C, C/A, or A/A. As described above, the genotype can comprise nucleotides at additional positions, such as positions in codons 49, 62, and 303, or any combination thereof.

Provided herein is a method of characterizing a Rabphillin-3A-Like gene in a subject comprising the steps of obtaining a biological sample from the subject, wherein the sample comprises a nucleic acid of human Rabphillin-3A-Like gene; amplifying the nucleic acid with a polymerase chain reaction-confronting two pair primer method (PCR-CTPP); wherein the PCR-CTPP method comprises contacting the nucleic acid with one or more primers comprising the sequence of GAGGGCACAGAGAACCTGTC (F1 primer) (SEQ ID NO:11), GGAGCACCCGGCTGGGGGTT (R1 primer) (SEQ ID NO:12), CATCTCAGATGTGACTCCCC (F21 primer) (SEQ ID NO:13), or GGCCCCAGAGGTACTCACTT (R2 primer) (SEQ ID NO:14); and identifying nucleotides at a polymorphic site in the 5′ untranslated region of exon 2 of the Rabphillin-gene. The identified nucleotide indicates the character of the polymorphic Rabphillin-3A-Like gene in the subject.

A number of methods are available for analyzing nucleic acids for the presence of a specific sequence. For all of the methods described herein, genomic DNA can be extracted from a sample and this sample can be from any organism and can be, but is not limited to, peripheral blood, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. Such samples can be obtained directly from a subject, commercially obtained or obtained via other means. Thus, the invention described herein can be utilized to analyze a nucleic acid sample that comprises genomic DNA, amplified DNA (such as a PCR product) cDNA, cRNA, a restriction fragment or any other desired nucleic acid sample. When one performs one of the herein described methods on genomic DNA, typically the genomic DNA will be treated in a manner to reduce viscosity of the DNA and allow better contact of a primer or probe with the target region of the genomic DNA. Such reduction in viscosity can be achieved by any desired methods, which are known to the skilled artisan, such as DNase treatment or shearing of the genomic DNA, preferably lightly.

If sufficient DNA is available, genomic DNA can be used directly. Alternatively, the region of interest is cloned into a suitable vector and grown in sufficient quantity for analysis. The nucleic acid may be amplified by conventional techniques, such as the polymerase chain reaction (PCR), to provide sufficient amounts for analysis. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers flanking any of the polymorphic sites of this invention.

Techniques useful herein include PCR-CTPP, DNA PCR followed by sequencing; sequence specific conformational polymorphism analysis; restriction fragment length polymorphism analysis; Allelic Discrimination by Real Time PCR (AD-RT-PCR). Optionally, the amplifying step comprises a polymerase chain reaction-confronting two pair primer method (PCR-CTPP). The primers used in the PCR-CTPP comprises one or more of the nucleic acid sequences selected from the group consisting of GAGGGCACAGAGAACCTGTC (F1 primer) (SEQ ID NO:11), GGAGCACCCGGCTGGGGGTT (R1 primer) (SEQ ID NO:12), CATCTCAGATGTGACTCCCC (F2 primer) (SEQ ID NO:13), and GGCCCCAGAGGTACTCACTT (R2 primer) (SEQ ID NO:14). One of skill in the art would know how to design primers accordingly to amplify any region of the gene and according to the amplification method selected.

Various methods are known in the art that utilize oligonucleotide ligation as a means of detecting polymorphisms, for examples see Riley et al (1990) Nucleic Acids Res 18:2887-2890; and Delahunty et al (1996) Am J Hum Genet. 58:1239-1246, which are incorporated herein by reference in their entirety for methods of detecting polymorphisms. Such methods include single base chain extension (SBCE), oligonucleotide ligation assay (OLA) and cleavase reaction/signal release (Invader methods, Third Wave Technologies

LCR and Gap LCR are exponential amplification techniques, both depend on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227, which is incorporated herein by reference in its entirety for the methods taught therein). Gap LCR (GLCR) is a version of LCR where the probes are not adjacent but are separated by 2 to 3 bases.

A method for typing single nucleotide polymorphisms in DNA, labeled Genetic Bit Analysis (GBA) has been described [Genetic Bit Analysis: a solid phase method for typing single nucleotide polymorphisms. Nikiforov T T; Rendle R B; Goelet P; Rogers Y H; Kotewicz M L; Anderson S; Trainor G L; Knapp M R. NUCLEIC ACIDS RESEARCH, (1994) 22 (20) 4167-75]. In this method, specific fragments of genomic DNA containing the polymorphic site(s) are first amplified by the polymerase chain reaction (PCR) using one regular and one phosphorothioate-modified primer. The double-stranded PCR product is rendered single-stranded by treatment with the enzyme T7 gene 6 exonuclease, and captured onto individual wells of a 96 well polystyrene plate by hybridization to an immobilized oligonucleotide primer. This primer is designed to hybridize to the single-stranded target DNA immediately adjacent from the polymorphic site of interest. Using the Klenow fragment of E. coli DNA polymerase I or the modified T7 DNA polymerase (Sequenase), the 3′ end of the capture oligonucleotide is extended by one base using a mixture of one biotin-labeled, one fluorescein-labeled, and two unlabeled dideoxynucleoside triphosphates. Antibody conjugates of alkaline phosphatase and horseradish peroxidase are then used to determine the nature of the extended base in an ELISA format. A semi-automated version of the method, which is called Genetic Bit Analysis (GBA), is being used on a large scale for the parentage verification of thoroughbred horses using a predetermined set of 26 diallelic polymorphisms in the equine genome. Additionally, minisequencing with immobilized primers has been utilized for detection of mutations in PCR products [Minisequencing: A Specific Tool for DNA Analysis and Diagnostics on Oligonucleotide Arrays. Pastinen, T. et al. Genome Research 7:606-614 (1997)].

The effect of phosphorothioate bonds on the hydrolytic activity of the 5′-->3′ double-strand-specific T7 gene 6 exonuclease in order to improve upon GBA was studied [The use of phosphorothioate primers and exonuclease hydrolysis for the preparation of single-stranded PCR products and their detection by solid-phase hybridization. Nikiforov T. Rendle R B; Kotewicz M L; Rogers Y H. PCR Methods and Applications, (1994) 3 (5) 285-91]. Double-stranded DNA substrates containing one phosphorothioate residue at the 5′ end were found to be hydrolyzed by this enzyme as efficiently as unmodified ones. The enzyme activity was, however, completely inhibited by the presence of four phosphorothioates. On the basis of these results, a method for the conversion of double-stranded PCR products into full-length, single-stranded DNA fragments was developed. In this method, one of the PCR primers contains four phosphorothioates at its 5′ end, and the opposite strand primer is unmodified. Following the amplification, the double-stranded product is treated with T7 gene 6 exonuclease. The phosphorothioated strand is protected from the action of this enzyme, whereas the opposite strand is hydrolyzed. When the phosphorothioated PCR primer is 5′ biotinylated, the single-stranded PCR product can be easily detected calorimetrically after hybridization to an oligonucleotide probe immobilized on a microtiter plate. A simple and efficient method for the immobilization of relatively short oligonucleotides to microtiter plates with a hydrophilic surface in the presence of salt can be used.

DNA analysis based on template hybridization (or hybridization plus enzymatic processing) to an array of surface-bound oligonucleotides is well suited for high density, parallel, low cost and automatable processing [Fluorescence detection applied to non-electrophoretic DNA diagnostics on oligonucleotide arrays. Ives, Jeffrey T.; Rogers, Yu Hui; Bogdanov, Valery L.; Huang, Eric Z.; Boyce-Jacino, Michael; Goelet, Philip L. L. C., Proc. SPIE-Int. Soc. Opt. Eng., 2680 (Ultrasensitive Biochemical Diagnostics), 258-269 (1996)]. Direct fluorescence detection of labeled DNA provides the benefits of linearity, large dynamic range, multianalyte detection, processing simplicity and safe handling at reasonable cost. The Molecular Tool Corporation has applied a proprietary enzymatic method of solid phase genotyping to DNA processing in 96-well plates and glass microscope slides. Detecting the fluor-labeled GBA dideoxynucleotides requires a detection limit of approximately. 100 mols/μm². Commercially available plate readers detect about 1000 mols./μm², and an experimental setup with an argon laser and thermoelectrically-cooled CCD can detect approximately 1 order of magnitude less signal. The current limit is due to glass fluorescence. Dideoxynucleotides labeled with fluorescein, eosin, tetramethylrhodamine, Lissamine and Texas Red have been characterized, and photobleaching, quenching and indirect detection with fluorogenic substrates have been investigated.

Other amplification techniques that can be used in the context of the present invention include, but are not limited to, Q-beta amplification as described in European Patent Application No 454-4610, strand displacement amplification as described in Walker et al. (1996) and EP A684 315 and, target mediated amplification as described in PCT Publication WO 9322461, the disclosures of which are incorporated herein by reference in their entirety for the methods taught therein.

Allele specific amplification can also be utilized for biallelic markers. Discrimination between the two alleles of a biallelic marker can also be achieved by allele specific amplification, a selective strategy, whereby one of the alleles is amplified without amplification of the other allele. For allele specific amplification, at least one member of the pair of primers is sufficiently complementary with a region of a gene sequence comprising the polymorphic base of a biallelic marker of the present invention to hybridize therewith. Such primers are able to discriminate between the two alleles of a biallelic marker. This can be accomplished by placing the polymorphic base at the 3′ end of one of the amplification primers. Such allele specific primers tend to selectively prime an amplification or sequencing reaction so long as they are used with a nucleic acid sample that contains one of the two alleles present at a biallelic marker because the extension forms from the 3′ end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well within the ordinary skill in the art.

A detectable label may be included in an amplification reaction or can be coupled with any of the nucleic acids disclosed herein. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., 32 P, 35 S, 3H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, can be analyzed by one of a number of methods known in the art. The nucleic acid can be sequenced by dideoxy or other methods. Hybridization with the variant sequence can also be used to determine its presence, by Southern blots, dot blots, etc. The hybridization pattern of a control (reference) and variant sequence to an array of oligonucleotide probes immobilized on a solid support, as described in U.S. Pat. No. 5,445,934 and WO95/35505, which are incorporated herein by reference in their entirety for the methods, may also be used as a means of detecting the presence of variant sequences. Single strand conformational polymorphism (SSCP) analysis, denaturing gradient gel electrophoresis (DGGE), mismatch cleavage detection, and heteroduplex analysis in gel matrices are used to detect conformational changes created by DNA sequence variation as alterations in electrophoretic mobility. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels.

The present invention also provides an array of oligonucleotides for identification of polymorphisms, where discrete positions on the array are complementary to one or more of the provided polymorphic sequences, e.g. oligonucleotides of at least 12 nt, frequently 20 nt, or larger, and including the sequence flanking the polymorphic position. Such an array may comprise a series of oligonucleotides, each of which can specifically hybridize to a different polymorphism of the present invention. Usually such an array will include at least 2 different polymorphic sequences, i.e. polymorphisms located at unique positions within the locus, and may include all of the provided polymorphisms. Therefore, the array can include sequences comprising the most commonly found allele at a position as well as other nucleotides found at this position. The array can optionally comprise the most commonly found allele at a second, third, fourth, fifth, or more positions as well as other nucleotides at each of these positions. Each oligonucleotide sequence on the array will usually be at least about 12 nt in length (i.e., 10-15 nt), may be the length of the provided polymorphic sequences, or may extend into the flanking regions to generate fragments of 100 to 200 nt in length.

The present invention also provides the use of the nucleic acid sequences of the invention in methods using a mobile solid support to analyze polymorphisms. See for example, WO 01/48244 which is incorporated herein by reference in its entirety for the methods taught therein. The method of performing a Luminex FlowMetrix-based SNP analysis involves differential hybridization of a PCR product to two differently-colored FACS-analyzable beads. The FlowMetrix system currently consists of uniformly-sized 5 micron polystyrene-divinylbenzene beads stained in eight concentrations of two dyes (orange and red). The matrix of the two dyes in eight concentrations allows for 64 differently-colored beads (82) that can each be differentiated by a FACScalibur suitably modified with the Luminex PC computer board. In the Luminex SNP analysis, covalently-linked to a bead is a short (approximately 18-20 bases) “target” oligodeoxynucleotide (oligo). The nucleotide positioned at the center of the target oligo encodes the polymorphic base. A pair of beads are synthesized; each bead of the pair has attached to it one of the polymorphic oligonucleotides. A PCR of the region of DNA surrounding the to-be analyzed SNP is performed to generate a PCR product. Conditions are established that allow hybridization of the PCR product preferentially to the bead on which is encoded the precise complement. In one format (“without competitor”), the PCR product itself incorporates a flourescein dye and it is the gain of the flourescein stain on the bead, as measured during the FACScalibur run, that indicates hybridization. In a second format (“with competitor”) the beads are hybridized with a competitor to the PCR product. The competitor itself in this case is labeled with flourescein. And it is the loss of the flourescein by displacement by unlabeled PCR product that indicates successful hybridization.

Each genotype described herein can be correlated with one or more clinical characteristics to generate a database of reference genotypes, such that one of skill in the art can compare a subject's genotype to a reference genotype or genotypes and determine whether the subject is predisposed to cancer, has cancer, responds well to a selected therapy, or has an invasive or aggressive type of cancer.

Since subjects will vary depending on numerous parameters including, but not limited to, race, age, weight, medical history etc., as more information is gathered on populations, the database can contain genotype information classified by race, age, weight, medical history etc., such that one of skill in the art can assess the subject's risk of developing cardiovascular disease based on information more closely associated with the subject's demographic profile. Where there is a differential distribution of a polymorphism by racial background or another parameter, guidelines for drug administration can be generally tailored to a particular group.

It will be appreciated by those skilled in the art that the nucleic acids provided herein as well as the nucleic acid sequences identified from subjects can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate a list of sequences comprising one or more of the nucleic acids of the invention. Another aspect of the present invention is a computer readable medium having recorded thereon at least 2, 5, 10, 15, 20, 25, 30, 50, 100, 200, 250, 300, 400, 500, 1000, 2000, 3000, 4000 or 5000 nucleic acids of the invention or nucleic acid sequences identified from subjects.

Thus, provided herein is a computer system comprising a database including records comprising a plurality of reference genotypes comprising the SNP in the 5′ UTR region of exon 2 of the Rabphillin-3A-Like gene and associated diagnosis, predisposition to disease, prognosis, therapy data, or any combination thereof; and a user interface capable of receiving a selection of one or more test genotypes for use in determining matches between the test genotypes and the reference genotypes and displaying the records associated with matching genotypes. The genotype in the data base can comprise nucleotides in the un-translated region of the Rabphillin-3A-Like gene and more precisely can comprises the nucleotides at position −25 of the 5′ untranslated region of the exon 2 of the Rabphillin-3A-Like gene.

Computer readable medium include magnetically readable media, optically readable media, electronically readable media and magnetic/optical media. For example, the computer readable medium may be a hard disc, a floppy disc, a magnetic tape, CD-ROM, DVD, RAM, or ROM as well as other types of other media known to those skilled in the art.

Embodiments of the present invention include systems, particularly computer systems which contain the sequence information described herein. As used herein, “a computer system” refers to the hardware components, software components, and data storage components used to store and/or analyze the nucleotide sequences of the present invention or other sequences. The computer system preferably includes the computer readable media described above, and a processor for accessing and manipulating the sequence data.

Preferably, the computer is a general purpose system that comprises a central processing unit (CPU), one or more data storage components for storing data, and one or more data retrieving devices for retrieving the data stored on the data storage components. A skilled artisan can readily appreciate that any one of the currently available computer systems are suitable.

In one particular embodiment, the computer system includes a processor connected to a bus which is connected to a main memory, preferably implemented as RAM, and one or more data storage devices, such as a hard drive and/or other computer readable media having data recorded thereon. In some embodiments, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In some embodiments, the data storage component is a removable computer readable medium such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device. Software for accessing and processing the nucleotide sequences of the nucleic acids of the invention (such as search tools, compare tools, modeling tools, etc.) may reside in main memory during execution.

In some embodiments, the computer system may further comprise a sequence comparer for comparing the nucleic acid sequences stored on a computer readable medium to another test sequence stored on a computer readable medium. A “sequence comparer” refers to one or more programs which are implemented on the computer system to compare a nucleotide sequence with other nucleotide sequences.

Accordingly, one aspect of the present invention is a computer system comprising a processor, a data storage device having stored thereon a nucleic acid of the invention, a data storage device having retrievably stored thereon reference nucleotide sequences to be compared with test or sample sequences and a sequence comparer for conducting the comparison. The sequence comparer may indicate a homology level between the sequences compared or identify a difference between the two sequences. For example, a reference sequence comprising SEQ ID NO:1 any fragment thereof can be compared with a test sequence from a subject to determine if the test sequence is the same as the reference sequence.

Alternatively, the computer program may be a computer program which compares a test nucleotide sequence(s) from a subject or a plurality of subjects to a reference nucleotide sequence(s) in order to determine whether the test nucleotide sequence(s) differs from or is the same as a reference nucleic acid sequence(s) at one or more positions. Optionally such a program records the length and identity of inserted, deleted or substituted nucleotides with respect to the sequence of either the reference polynucleotide or the test nucleotide sequence. In one embodiment, the computer program may be a program which determines whether the nucleotide sequences of the test nucleotide sequence contains one or more single nucleotide polymorphisms (SNP) with respect to a reference nucleotide sequence. These single nucleotide polymorphisms may each comprise a single base substitution, insertion, or deletion.

Accordingly, another aspect of the present invention is a method for determining whether a test nucleotide sequence differs at one or more nucleotides from a reference nucleotide sequence comprising the steps of reading the test nucleotide sequence and the reference nucleotide sequence through use of a computer program which identifies differences between nucleic acid sequences and identifying differences between the test nucleotide sequence and the reference nucleotide sequence with the computer program.

The computer program can be a program which identifies single nucleotide polymorphisms. The method may be implemented by the computer systems described above. The method may also be performed by reading at least 2, 5, 10, 15, 20, 25, 30, 50, 100, or more test nucleotide sequences and the reference nucleotide sequences through the use of the computer program and identifying differences between the test nucleotide sequences and the reference nucleotide sequences with the computer program. A computer program that identifies single nucleotide polymorphisms in the gene sequence and determines a subject's genotype is also contemplated by this invention. This invention also provides for a computer program that correlates genotypes with clinical status such that one of skill in the art can assess a subject's risk of developing cancer, likelihood of having aggressive or invasive cancer, etc. The computer program can optionally include treatment options or drug indications for subjects with specific genotypes.

The nucleic acids of the invention (both test nucleic acid sequences and reference nucleic acid sequences) may be stored and manipulated in a variety of data processor programs in a variety of formats. For example, they may be stored as text in a word processing file, such as MicrosoftWORD® or WORDPERFECT® or as an ASCII file in a variety of database programs familiar to those of skill in the art, such as DB2®, SYBASE®, or ORACLE®. In addition, many computer programs and databases may be used as sequence comparers, identifiers, or sources of reference nucleotide sequences. The following list is intended not to limit the invention but to provide guidance to programs and databases which are useful with the nucleic acid sequences of the invention. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, 1990), FASTA (Pearson and Lipman, 1988), FASTDB (Brutlag et al., 1990), Catalyst (Molecular Simulations Inc.), Catalyst/SHAPE (Molecular Simulations Inc.), Cerius.sup.2.DBAccess (Molecular Simulations Inc.), HypoGen (Molecular Simulations Inc.), Insight II (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, the BioByteMasterFile database, the Genbank database, and the Genseqn database. Many other programs and data bases would be apparent.

The following examples are put forth so as to provide those of ordinary skill in the art with a complete disclosure and description of how the antibodies, polypeptides, nucleic acids, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

### EXAMPLES

### Example 1

This study was focused on identification and characterization of different novel genes and gene products involved in the development and progression of colorectal cancer (CRC) and in assessing their clinical utility to predict the therapeutic responses and/or patient survival. The human chromosome region 17p shows frequent allelic losses/mutations in CRCs. Such genetic alterations are a hall mark for the presence of tumor suppressor genes and suggest for the existence of additional tumor suppressor genes besides p53, which is known to occur at 17p13.1 locus. In a recent study, the clinical utility of abnormalities in p53 was evaluated. The abnormal phenotypic expression is a useful prognostic marker particularly for non-Hispanic Caucasian patients and particularly for tumors located in the proximal colon.

Previously, Batra et al. (Batra, S. K., McLendon, R. E., Koo, J. S., Castelino-Prabhu, S., Fuchs, H. E., Krischer, J. P., Friedman, H. S., Bigner, D. D., and Bigner, S. H. Prognostic implications of chromosome 17p deletions in human medulloblastomas. J Neurooncol, 24: 39-45, 1995) reported an association between a hemizygous deletion of 17p13.3 and the clinical outcome in human medulloblastoma and suggested that patients with deletions in this chromosomal region had poor survival. Subsequently, Smith et al. (Smith, J. S., Tachibana, I., Allen, C., Chiappa, S. A., Lee, H. K., McIver, B., Jenkins, R. B., and Raffel, C. Cloning of a human ortholog (RPH3AL) of (RNO)Rph3a1 from a candidate 17p13.3 medulloblastoma tumor suppressor locus. Genomics, 59: 97-101, 1999) identified the RPH3AL gene at the 17p13.3 locus (GenBank #AF129812) and suggested it as a human ortholog of the rat Rabphillin-3A-like gene (Rph3a1). Smith et al. also cloned, sequenced and performed mutational analysis of RPH3AL in medullobastoma, follicular thyroid carcinoma as well as ovarian carcinoma specimens. The studies of Smith et al. failed to identify any missense mutations in RPH3AL; therefore, they concluded that RPH3AL was not involved in the oncogenesis of these neoplasms (Smith, J. S., Tachibana, I., Allen, C., Chiappa, S. A., Lee, H. K., McIver, B., Jenkins, R. B., and Raffel, C. Cloning of a human ortholog (RPH3AL) of (RNO)Rph3a1 from a candidate 17p13.3 medulloblastoma tumor suppressor locus. Genomics, 59: 97-101, 1999). In addition, polymorphisms in the untranslated regions (UTRs) of several human genes have previously been implicated in mRNA transcription, message stability or expression, as seen in thymidylate synthase (TS) (Zhang, J., Cui, Y., Kuang, G., Li, Y., Wang, N., Wang, R., Guo, W., Wen, D., Wei, L., Yu, F., and Wang, S. Association of the thymidylate synthase polymorphisms with esophageal squamous cell carcinoma and gastric cardiac adenocarcinoma. Carcinogenesis, 25: 2479-2485, 2004) and CYP17 (Nedelcheva Kristensen, V., Haraldsen, E. K., Anderson, K. B., Lonning, P. E., Erikstein, B., Karesen, R., Gabrielsen, O, S., and Borresen-Dale, A. L. CYP17 and breast cancer risk: the polymorphism in the 5′ flanking area of the gene does not influence binding to Sp-1. Cancer Res, 59: 2825-2828, 1999).

All exonic, both coding and non-coding, regions of the RPH3AL gene in prospectively collected primary sporadic CRC samples were analyzed. This analysis identified several novel missense mutations of RPH3AL in CRCs, and specifically demonstrated that a single nucleotide polymorphism (SNP) at the −25 position (Cytosine to Adenine) in the 5′ untranslated region in the exon 2 of this gene (5′UTR-25) had a strong association with regional lymph node invasion, distant metastasis and poor patient survival.

Clinical Information and Tissues.

Patient clinical information was retrieved retrospectively in a blinded fashion from the medical charts as well as from the UAB-Tumor Registry. Patients were followed by the UAB tumor registry until their death or the date of the last documented contact within the study time frame. The tumor registries ascertain outcome (mortality) information directly from patients (or living relatives) and from the physicians of the patients through telephone and mail contacts. This information is further validated against State Death Lists. Demographic data including patient age at diagnosis, gender, race/ethnicity, date of surgery, data of the last follow-up (if alive), date of death and pathologic features, tumor stage, differentiation, mucin content nodal involvement etc were collected. The tumor registries update follow-up information every six months and follow-up of the retrospective cohort ended in January 2005.

Prospective CRC Samples

Tissue samples from 95 consecutive, unselected patients with histologically confirmed CRC and corresponding normal (benign epithelium) tissues were collected fresh at surgery, snap-frozen in liquid nitrogen, and stored at −80° C. until analyzed. All patients had undergone surgical resection for first primary CRC from January 1996 through December 2004; however, the majority of these cases were from years 2002 through 2004.

Retrospective CRC Samples.

Additionally, randomly selected 134 archival tissues from CRC patients who had undergone surgical resection for first primary CRC from 1981 through 1993 were collected. Formalin fixed paraffin embedded tissue blocks of these patients were obtained from the Anatomic Pathology Division of UAB. The tissues were analyzed only to assess the status of single nucleotide polymorphism at −25 untranslated region at 5′ end of the RPH3AL gene (5′UTR-25).

The clinical, demographic and pathologic information for these retrospective cases were obtained as described above. During the initial selection process, those patients who died within a week of their surgery, those patients with surgical margin-involvement, or unspecified tumor location, or multiple primaries within the colorectum, or multiple malignancies, or those patients with family or personal history of CRC were all excluded from the study population. However, based on the information in patient charts, it will be difficult to identify the familial vs. sporadic nature of the tumors, therefore, this retrospective cohort can be described as ‘consecutive’ population of patients with Stages I, II, III and IV CRCs. Follow-up period this retrospective cohort population ranges from <1 to >18 years.

CRC Tumor Analysis

Before utilizing the tissues for sequence analysis, a frozen section was taken to assess the proportion of tumor versus uninvolved tissue in the sample and to permit microdissection (using simple microscope) to separate tumor from uninvolved tissue. In addition, paraffin tissue sections were utilized to assess the phenotypic expression of several other markers described below.

Molecular Analysis of the RPH3AL Gene.

Using reverse transcription-polymerase chain reaction technique (RT-PCR) and DNA sequencing methods, 95 frozen specimens of CRCs and matching benign colonic epithelial tissues were analyzed. Direct DNA sequencing method using RPH3AL specific primers (forward 5′ to 3′: GTGCACTTTGGAGACAGCAA and reverse 5′ to 3′: GTGGGAGGGGAGGGTAATAA) (SEQ ID NOS: 15 and 16, respectively) resulted in a cDNA transcript which covers exons 1 though 9. The specific primers were designed using the LASERGENE (DNAStar) Primer design software. The initial study on RPH3AL (Smith, J. S., Tachibana, I., Allen, C., Chiappa, S. A., Lee, H. K., McIver, B., Jenkins, R. B., and Raffel, C. Cloning of a human ortholog (RPH3AL) of (RNO)Rph3a1 from a candidate 17p13.3 medulloblastoma tumor suppressor locus. Genomics, 59: 97-101, 1999) suggested that the putative start codon was identified in the middle of the exon 2; thus the cDNA transcript generated in this study covers the untranslated region of 5′ end (exon 1 through˜the first half of exon 2). As such, the cDNA transcript covers the untranslated regions of exons 1 and 2.

RNA Extraction, PCR Analysis, and DNA Sequencing of RPH3AL.

Total cytoplasmic RNA was extracted directly from frozen tissues with a RNeasy Kit (QIAGEN). Total RNA was used as template in oligo (dT)-primed first strand cDNA synthesis with SuperScript RT (Life Technologies). One-tenth of the first-strand cDNA synthesis reaction was used as template in PCR with the RPH3AL forward (5′-GTGCACTTTGGAGACAGCAA-3′) (SEQ ID NO:15) and reverse (5′-GTGGGAGGGGAGGGTAATAA-3′) (SEQ ID NO: 16) amplification primers. Thermostable proofreading DNA polymerase rTth XL (Perkin-Elmer) was used for the RPH3AL status. Mutation analysis of RPH3AL was performed by direct sequencing of the RPH3AL cDNAs amplified in RT-PCR, the appropriate reaction products were used as template in cycle-sequencing reactions (Perkin-Elmer) with RPH3AL-sequencing primers. Sequencing reaction products were resolved on an ABI Prism 307 Automated DNA sequencer (UAB Comprehensive Cancer Center Sequencing Facility). Compilation and sequence analyses was performed using the LASERGENE (DNAStar) sequence analysis software, which allowed for direct analysis of sequencing eloctrophoretograms for the detection of duplex sequence signals at each position to identify mutations/polymorphisms. Nucleotide changes in each cDNA sequence were confirmed by sequencing both the strands.

Genotyping of the single nucleotide polymorphism at UTR-25.

Genomic DNA extracted from CRCs and the matching benign colonic epithelial frozen as well as archival specimens were analyzed for the genotype of the C/A SNP at 5′UTR-25 utilizing the PCR-confronting two pair primer (PCR-CTPP) method as described below.

PCR-Confronting Two Pair Primer (PCR-CTPP) Method.

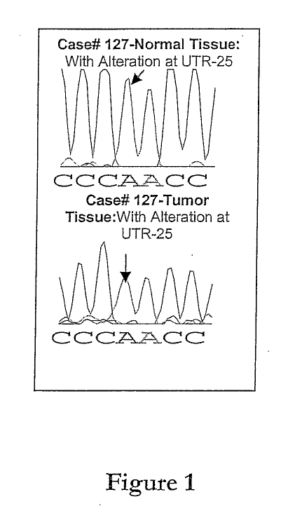

The PCR-CTPP is a genotyping method that can be applied to most single-nucleotide variations (Hamajima, N., Saito, T., Matsuo, K., and Tajima, K. Competitive amplification and unspecific amplification in polymerase chain reaction with confronting two-pair primers. J Mol Diagn, 4: 103-107, 2002). The amplification of allele-specific bands of different lengths by using four primers enables genotyping by electrophoresis without other steps. As shown in FIG. 1, the four primers used to analyze the genotype of the C/A SNP at 5′UTR-25, consist of F1 and R1 for the amplification of one allele, and F2 and R2 for the amplification of the other allele. F1 and R2 produce a common PCR product that is independent of the difference in alleles. F2 and R1 confront each other at the 3′ end with the base specific to the allele. The primers designed to detect the UTR-25 C>A variant were: F1 (5′-GAGGGCACAGAGAACCTGTC-3′) (SEQ ID NO:11), R1 (5′-GAGCACCCGGCTGGGGTT-3′) (SEQ ID NO:12), F2 (5′-CATCTCAGATGTGACTCCCC-3′) (SEQ ID NO:13), and R2 (5′-GGCCCCAGAGGTACTCACTT-3′) (SEQ ID NO:14). The 25 L reaction mixture consisted of 10×PCR buffer, 50 mM of each dNTP, 10 pmoles of each primer, 0.5 units of Platinum Taq Polymerase (Invitrogen), and 100 ng of genomic DNA. Amplification was achieved by 5 minutes of initial denaturation at 94° C. followed by 35 cycles of 30 seconds at 94° C., 30 seconds at 60° C. and 1 minute at 70° C. and a 7 minutes final extension at 70° C. PCR products were fractionated by electrophoresis with a 2% agarose gel and stained with ethidium bromide. The products of PCR-CTPP were analyzed by direct DNA sequencing to reconfirm the genotype of 5′UTR-25.

Statistical Analysis.

The χ²-test was used to compare baseline characteristics as described in Fleiss, J. [Statistical methods for rates and proportions. New York, N.Y.: John Wiley and Sons, 1981]. Recurrence of CRC (local recurrence or distant metastases) and deaths due to CRC were the outcomes (events) of interest. The predictive and prognostic significance of SNP at 5′UTR-25 was analyzed using Kaplan-Meier as described in Kaplan, E. and Meier, P. [Non-parametric estimation from incomplete observations. J Am Stat Assoc, 53, 1958]. and Cox proportional hazards regression analysis methods [Cox, D. R. Regression models and life tables. J Roy Stat Soc, 34: 187-220, 1972]. Demographic variables included in the analysis were age (<65 and ≧65 years), gender, and ethnicity. Pathological variables included pT (depth of tumor invasion), pN (nodal involvement), M (distant metastasis), tumor differentiation (low or high grade), tumor size (≦5 cm and >5 cm) in maximal dimension, and tumor location (proximal colon or distal colorectum). For recurrence analyses, the time at risk was measured by calculating the number of months from date of surgery to time of recurrence. Patients who had a recurrence of their CRC were identified and classified as “event”, while the remaining patients without recurrence, 1) who died due to their CRC, or 2) died due to causes other than CRC, or 3) who were alive at the end of the follow-up period were “right censored”. For survival analyses, the risk of CRC-specific death was measured by calculating the number of months from the date of surgery to death or the date of last contact. Patients who died of a cause other than CRC or who were alive at the end of the follow-up period were “right censored”.

The log-rank test was used to compare Kaplan-Meier curves based on the genotype status of SNP at 5′UTR-25. The Kaplan-Meier estimates were also used to obtain recurrence rates or survival probability at 5 year after surgery. Separate multivariate Cox regression models were built to assess the value of the type of genotype at 5′UTR-25 of the RPH3AL in predicting disease recurrence and patient survival. We controlled for all demographic and clinicopathological variables described above in these multivariate analyses. All analyses were performed with SAS statistical software version 9.1 (Allison, P. Survival Analysis Using the SAS System: A Practical Guide. Cary, N.C.: SAS Institute Inc, 1995; Kleinbaum, D. Survival Analysis: A Self Learning Text. New York, N.Y.: Springer-Verlag, 1996). P values were calculated and significance was analyzed at an alpha level of 0.05.

Mutational Status of the Rabphillin-3A-Like Gene in CRCs.

Only frozen CRC specimens (n=95) were analyzed for the mutational status of the RPH3AL utilizing the molecular approaches described above (RT-PCR); and the findings are presented in Table 1. Table 1 shows the mutational analysis of the Rabphillin-3A-like gene in prospectively collected CRCs. (UTR=Untranslatable Region; Wt=Wild-type; Neg=Negative; Pos=Positive; AA=African-Americans; W=Caucasians; M=Male; F=Female; PC=Proximal Colon; DC=Distal Colorectum; H=High; L=Low; SNPs=All single nucleotide polymorphisms without co-existence of MM; MM=Total missense mutations; Total=includes all cases with polymorphisms in translatable area without co-existence of missense point mutations and cases with MM; SNPs at codon 49, 62 and 303 are in the translatable region of RPH3AL.)

As shown in Table 1, 49 of 95 (52%) CRCs exhibited genetic alterations in the RPH3AL gene; while 43 of these mutations were SNPs and the remaining 6 were missense point mutations. Thirty six of the 43 SNPs (36 of 95, 38%, C/C>C/A or A/A) were detected at the 5′UTR-25 position in the exon 2 of RPH3AL (Table 1). The distribution of missense point mutations of RPH3AL is shown in Table 2. Table 2 also shows the coexistence of missense point mutations of RPH3AL and p53 genes in CRCs.

Two CRCs exhibited missense mutations at codon 67, the remaining 4 CRCs demonstrated mutations at codons 98, 175, 268, or 290. Mutations at codon 67, 175, 268 or 290 were missense point mutations resulting in amino acid substitutions and all these residues were non-conservative type; whereas, alteration at codon 98 was a silent mutation (transition).

The SNP at 5′UTR-25 resulted in a change in the nucleotide from Cytosine to Adenine (transversion) and it was observed both in tumor and its matching benign colonic epithelial tissue; thus, it can be considered as a polymorphism (FIG. 1).

TABLE 1
Mutational analysis of the Rabphillin-3A-like gene in prospectively collected CRCs

| | | | | Age | | | | | | | | | | | |
|---|---|---|---|---|---|---|---|---|---|---|---|---|---|---|---|
| | | | Amino | group | | | | | | | | Tumor | | | |
| RPH3AL | Total # | Nucleic Acid | Acid | (yrs) | | Race | | Gender | | Nodal Status | | Location | | Tumor Size | |
| Alterations | N (%) | Wt > Altered | Wt > Altered | <65 | ≧65 | AA | W | F | M | Neg | Pos | PC | DC | ≦5 cm | >5 cm |
| Total | 95 | — | — | 39 | 56 | 39 | 56 | 43 | 52 | 47 | 47 | 53 | 41 | 43 | 45 |
| Wild | 46 (48) | Wt | Wt | 21 | 25 | 27 | 19 | 25 | 21 | 26 | 20 | 30 | 16 | 17 | 24 |
| Type | | | | | | | | | | | | | | | |
| UTR-25 | 36 (38) | −25 (C > A) | Exon 2 | 15 | 21 | 6 | 30 | 12 | 24 | 19 | 17 | 14 | 21 | 18 | 17 |
| 62 | 3 (3) | GCA > GCC | Ala > Ala | 0 | 3 | 1 | 2 | 0 | 3 | 0 | 3 | 0 | 3 | 2 | 0 |
| 49/62 | 3 (3) | CCG > CCT/ | Pro > Pro/ | 1 | 2 | 1 | 2 | 1 | 2 | 0 | 2 | 1 | 2 | 2 | 1 |
| | | GCA > GCC | Ala > Ala | | | | | | | | | | | | |
| 62/67 | 2 (2) | GCA > GCC/ | Ala > Ala | 1 | 1 | 1 | 1 | 1 | 1 | 0 | 2 | 2 | 0 | 1 | 1 |
| | | GTC > ATC | Val > Ile | | | | | | | | | | | | |
| 49/62/175 | 1 (1) | CCG > CCT/ | Pro > Pro/ | 1 | 0 | 1 | 0 | 1 | 0 | 1 | 0 | 0 | 1 | 1 | 0 |
| | | GCA > GCC/ | Ala > Ala/ | | | | | | | | | | | | |
| | | CCC > TCC | Pro > Ser | | | | | | | | | | | | |
| 98 | 1 (1) | TGC > TGT | Cys > Cys | 0 | 1 | 1 | 0 | 0 | 1 | 0 | 1 | 0 | 1 | 0 | 1 |
| 268 | 1 (1) | GCC > GGC | Ala > Gly | 0 | 1 | 0 | 1 | 1 | 0 | 1 | 0 | 0 | 1 | 0 | 1 |
| 290 | 1 (1) | AGG > AAG | Arg > Lys | 0 | 1 | 1 | 0 | 1 | 0 | 0 | 1 | 0 | 1 | 1 | 0 |
| 303 | 1 (1) | GCT > GTT | Ala > Val | 0 | 1 | 0 | 1 | 1 | 0 | 0 | 1 | 1 | 0 | 1 | 0 |
| SNPs | 43 (45) | — | — | 16 | 27 | 8 | 35 | 14 | 29 | 19 | 23 | 16 | 26 | 23 | 18 |
| All MM | 6 (6) | — | — | 2 | 4 | 4 | 2 | 4 | 2 | 2 | 4 | 2 | 4 | 3 | 3 |
| Total | 49 (52) | — | — | 18 | 31 | 12 | 37 | 18 | 31 | 21 | 27 | 18 | 30 | 26 | 21 |

UTR = Untranslatable Region;
Wt = Wild-type;
Neg = Negative;
Pos = Positive;
AA = African-Americans;
W = Caucasians;
M = Male;
F = Female;
PC = Proximal Colon;
DC = Distal Colorectum;
H = High;
L = Low;
SNPs = All single nucleotide polymorphisms without co-existence of MM;
MM = Total missense mutations;
Total = includes all cases with polymorphisms in translatable area without co-existence of missense point mutations and cases with MM; SNPs at codon 49, 62 and 303 are in the translatable region of RPH3AL.

TABLE 2
CRC Patients exhibiting missense mutations in the RPH3AL gene also exhibited missense mutations in the p53 gene

| | RPH3AL Missense Mutations* | | | | p53 gene Missense Mutations | | | | Nodal & Distant |
|---|---|---|---|---|---|---|---|---|---|
| | | | Amino | | | | Amino | | Metastasis and |
| Case | Codon | Nucleotide | Acid | Types of | Codon | Nucleotide | Acid | Types of | Tumor |
| No. | No. | Altered | Altered | Alteration | No. | Altered | Altered | Alteration | Differentiation |
| 75 | 67 | GTC > ATC | Val > Ile | Transition | 175 | CGC > CAC | Arg > His | Transition | N+, M₀, Poor |
| 166 | 67 | GTC > ATC | Val > Ile | Transition | 135 | TGC > TAC | Cys > Tyr | Transition | N+, M₀, Poor |
| 128 | 98 | TGC > TGT | Cys > Cys | Transition | 248 | CGG > CAG | Arg > Gln | Transition | N+, M₀, Mod |
| 209 | 175 | CCC > TCC | Pro > Ser | Transition | 273 | CGT > CAT | Arg > His | Transition | N−, M₀, Well |
| 168 | 268 | GCC > GGC | Ala > Gly | Transversion | 236 | TAC > TGC | Tyr > Cys | Transition | N−, M₀, Mod |
| 183 | 290 | AGG > AAG | Arg > Lys | Transition | 258 | GAA > GAC | Glu > Asp | Transversion | N+, M₀, Poor |

*All these mutations are novel and were not reported earlier;
N− = Nodal metastasis Negative;
N+ = Nodal metastasis Positive;
M₀ = No metastasis to distant organs like liver;
Mod = Moderate Differentiation.

### Genotyping of the SNP at 5′UTR-25.

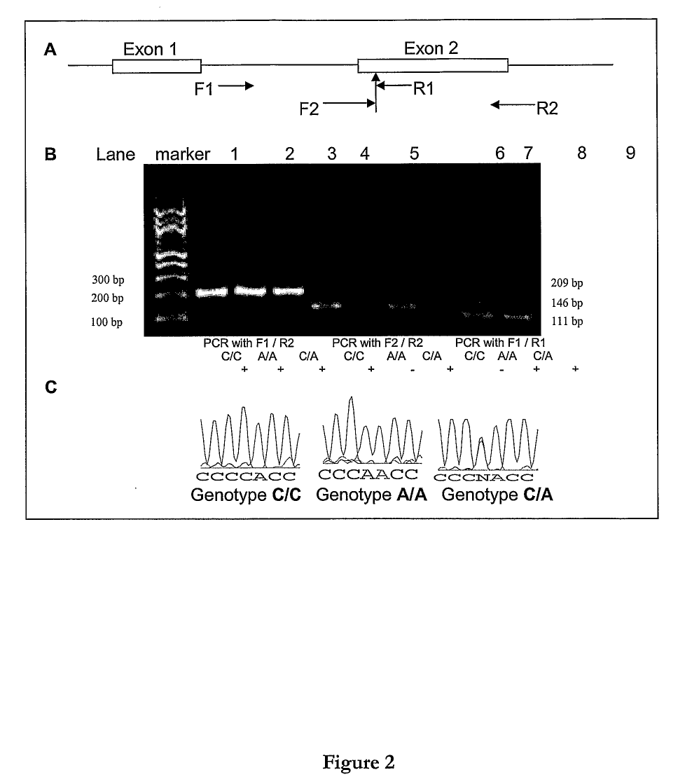

The genotype at 5′UTR-25 of RPH3AL was examined by following standard RT-PCR, direct sequencing or PCR-CTPP methods in tumors as well as corresponding normal tissues (FIG. 2). Incidence and frequencies of 5′UTR-25 genotypes of both patient cohorts (prospective as well as retrospective samples; N=229) are illustrated in Table 3. Specifically, Table 3 shows the genotypes of RPH3AL at UTR-25 and their correlations with different demographic and pathologic features. (UTR=−25 of 5′-Untranslatable Region; Neg=Node Negative; Pos=Node Positive; AA=African-Americans; W=Non-Hispanic Caucasians; M=Male; F=Female; PC=Proximal Colon; DC=Distal Colorectum; cm=Centimeters H=High; L=Low; *=Nodal status and Tumor location not available in one case. **=Tumor grade not available on 11 cases. ***=Tumor size data is not available on seven cases.)

Analysis of 95 prospectively collected frozen tissues for the genotype at 5′UTR-25 demonstrated that the genotype C/C, A/A and C/A, was found in 59 (62%), 9 (10%) and 27 (28%) CRCs, respectively. A similar pattern of genotype frequencies at 5′UTR-25 in 84 retrospective CRC samples were observed and the frequencies of C/C, A/A and C/A were 43 (51%), 8 (10%) and 33 (39%), respectively (Table 3).

Clinical Correlation of 5′UTR-25 Alterations in RPH3AL Gene.

The distribution of SNPs at 5′UTR-25 is demonstrated in Table 4. In total, 95% (19 of 20) of all prospective and retrospective cases with the genotype of A/A at 5′UTR-25 were associated with nodal involvement; in contrast, only 31% (23 of 75) cases with the genotype of C/A exhibited nodal involvement. CRCs with the genotype C/C were evenly distributed into node positive (51%) and node negative (49%) categories (χ² P<0.0001). Among the cases with the genotype A/A, the majority of patients were males (14 of 20, χ² P=0.006) and all of them were non-Hispanic Caucasians (20 of 20, Fisher Exact P<0.0001), and their tumors invaded into the deeper layers of the bowel wall (pT component of the TNM staging) (20 of 20, Fisher exact P=0.01). The majority of CRCs with the A/A variant genotype were larger than or equal to 5 cm in size (13 of 19, χ² P=0.06); however, there was no association between the type of genotype at 5′UTR-25 and either tumor grade or tumor location (Table 4). Because no statistically significant correlations were observed between the clinicopathological features and the missense mutations or SNPs of RPH3AL other than SNP at 5′UTR-25, genetic alterations other than 5′UTR-25 were not considered in further analyses.

TABLE 3
Genotypes of RPH3AL at UTR-25 and their correlations with different demographic and pathologic features

| Total# | Age group | | | | | | Nodal | | Tumor | | Tumor | | Tumor | |
|---|---|---|---|---|---|---|---|---|---|---|---|---|---|---|
| N | (years) | | Race | | Gender | | Status* | | Location* | | Grade** | | Size*** | |
| (%) | <65 | ≧65 | AA | W | F | M | Neg | Pos | PC | DC | L | H | ≦5 cm | >5 cm |

Prospectively collected Frozen Tissues

| | | | | | | | | | | | | | | | |
|---|---|---|---|---|---|---|---|---|---|---|---|---|---|---|---|
| Total | 95 | 39 | 56 | 39 | 56 | 43 | 52 | 47 | 47 | 53 | 41 | 61 | 23 | 43 | 45 |
| UTR-25: | 59 (62) | 24 | 35 | 33 | 26 | 31 | 28 | 28 | 30 | 39 | 20 | 36 | 18 | 25 | 28 |
| C/C | | | | | | | | | | | | | | | |
| UTR-25: | 9 (10) | 5 | 4 | 0 | 9 | 3 | 6 | 0 | 9 | 5 | 4 | 6 | 2 | 2 | 7 |
| A/A | | | | | | | | | | | | | | | |
| UTR-25: | 27 (28) | 10 | 17 | 6 | 21 | 9 | 18 | 19 | 8 | 9 | 17 | 19 | 3 | 16 | 10 |
| C/A | | | | | | | | | | | | | | | |

Retrospectively collected Archival Paraffin Blocks

| | | | | | | | | | | | | | | | |
|---|---|---|---|---|---|---|---|---|---|---|---|---|---|---|---|
| Total | 84 | 31 | 53 | 30 | 54 | 34 | 50 | 44 | 40 | 41 | 43 | 38 | 46 | 47 | 37 |
| UTR-25: | 43 (51) | 17 | 26 | 25 | 18 | 24 | 19 | 21 | 22 | 21 | 22 | 17 | 26 | 24 | 19 |
| C/C | | | | | | | | | | | | | | | |
| UTR-25: | 8 (10) | 2 | 6 | 0 | 8 | 2 | 6 | 0 | 8 | 3 | 5 | 5 | 3 | 2 | 6 |
| A/A | | | | | | | | | | | | | | | |
| UTR-25: | 33 (39) | 12 | 21 | 5 | 28 | 8 | 25 | 23 | 10 | 17 | 16 | 16 | 17 | 21 | 12 |
| C/A | | | | | | | | | | | | | | | |

UTR = −25 of 5′-Untranslatable Region;
Neg = Node Negative;
Pos = Node Positive;
AA = African-Americans;
W = Non-Hispanic Caucasians;
M = Male;
F = Female;
PC = Proximal Colon;
DC = Distal; Colorectum;
cm = Centimeters
H = High;
L = Low;
*= Nodal status and Tumor location not available in one case.
**= Tumor grade not available on 11 cases.
***= Tumor size data is not available on seven cases.

Genotype of RPH3AL at 5′UTR-25 and Patient Survival

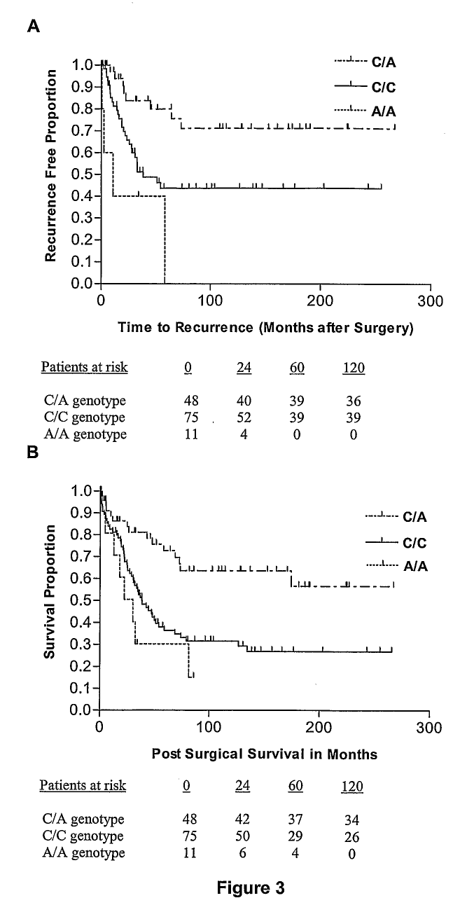

Kaplan-Meier univariate analysis demonstrated that patients homozygous for the A allele or C allele at 5′UTR-25 had higher risk of recurrence within 5 years after surgery compared to patients who were heterozygous for the C/A alleles (log-rank P<0.0001 and P=0.008, respectively) (FIG. 3A). However; there was no significant difference in risk of recurrence between patients homozygous for the A and C alleles (log-rank, P=0.10) (FIG. 3A).

Multivariate Cox Proportional Hazards analysis for disease recurrence demonstrated that patients with A/A variant genotype had a 13.85 times higher risk of CRC recurrence within 5 years of surgery compared to patients with the C/A variant genotype (CI: 3.12-16.43). Patients with the C/C variant genotype had a 4.64 times higher risk of CRC recurrence within 5 years of surgery compared to patients with the C/A variant genotype (CI: 1.65-13.07); whereas, there was no significant difference in risk of recurrence among patients with A/A and C/C variant genotypes, when adjusted for all demographic and clinicopathological features as shown in Table 5. The analyses have also demonstrated that patients with nodal metastasis were 2.41 times more likely to have a recurrence within 5 years of surgery compared to those without nodal metastasis (CI: 1.12-5.18) when adjusted for all other variables (Table 5).

Survival Analyses

Kaplan-Meier univariate survival analysis of the retrospective CRC cohort demonstrated that patients homozygous for the A allele at 5′UTR-25 had a significantly poorer 5-year survival as compared to patients heterozygous for the C/A alleles (log-rank P<0.001) (FIG. 3B). There was also a significant difference in the 5-year survival of patients homozygous for the C allele compared to patients heterozygous for the C/A alleles (log-rank, P<0.001). However, there was no significant difference in survival between patients homozygous for the A or C alleles (log-rank, P=0.21) (FIG. 3B).

Multivariate Cox proportional hazards analyses demonstrated that patients with the A/A variant genotype were 3.88 times more likely to die due to CRC within 5 years post surgery as compared to patients with the C/A variant genotype (CI: 1.39-10.82); whereas, there was no significant difference in the risk of death for patients with genotypes C/C vs. C/A or A/A vs. C/C when adjusted for all demographic and clinicopathological features (Table 5). Patients with nodal involvement were 2.25 times more likely to die due to CRC compared to those who were node negative (CI: 1.30-3.92) while patients with high tumor grade were 1.95 times more likely to die due to CRC as compared to those with low tumor grade in the 5 years post surgical period when adjusted for all other features (CI: 1.14-3.31) (Table 5).

### Example 2

### Association Between RPH3AL and p53 Tumor Suppressor Gene

It has been reported that there was a positive association between p53 mutational status and RPH3AL gene mutational status and suggested that alterations in RPH3AL gene combined with alterations of the p53 gene may be involved in tumor development, proliferation and differentiation [Goi, T., Takeuchi, K., Katayama, K., Hirose, K. and Yamaguchi, A. Mutations of rabphillin-3A-like gene in colorectal cancers. Oncol Rep, 9: 1189-1192, 2002].

RT-PCR and Direct DNA Sequencing for RPH3AL and p53

RNA was extracted from frozen tissues by using RNAeasy mini kit (QIAGEN, Hilden, Germany). cDNA was prepared from 10 ng/μl of purified RNA adding 200 units/μl of superscript III (Invitrogen Life Technologies, Carlsbad, Calif.), 4 μl of 5×RT buffer, 1 μl of 10 mM dNTP mix, 1 μl of 50 μm Oligo dT, 2 μl of 0.1 M DTT, 4 μl of 25 mM Mgcl2, and 40 units/μl of RNase OUT. Reverse transcription was performed by incubating samples for 50 min at 50° C. and then heating at 70° C. for 15 min to inactivate superscript III. The RNA template from cDNA-RNA hybrid molecule was removed by digesting with RNaseH on incubation at 37° C. for 20 min. The final volume of the cDNA reaction mixture was 25 μl. Five micro liters of cDNA was used to amplify p53 gene and RPH3AL gene separately by PCR (Polymerase Chain Reaction) adding 0.5 μl of 10 mM dNTPs, 2.5 μl of 10× Fast Start buffer plus 2 mM Mgcl2, 0.4 μl of 5 units/μl Fast start Taq DNA polymerase (Roche), 15.6 μl of nuclease free water (Promega), and a set of gene specific primers (each 10 pmoles/0.5 μl). A forward primer (p53A5F) 5′-GCTTTCCACGACGGTGAC-3′ (SEQ ID: 17) and a reverse primer (p53CIR) 5′-ACCCAAAACCCAAAATGGCAG-3′ (SEQ ID: 18) were used to cover entire coding region of p53 gene (exons 2 to 11) including part of 5′ and part of 3′ untranslated regions. A forward primer (RPH3ALVF) 5′-GTGCACTTTGGAGACAGCAA-3′ (SEQ ID: 15) and a reverse primer (RPH3ALVR) 5′-GTGGGAGGGGAGGGTAATAA-3′ (SEQ ID:16) were used to cover entire coding region of RPH3AL gene (exons 1 to 9) including part of 5′ and part of 3′ untranslated regions. Total 25 μl reaction mix was incubated on Robocycler (Strategene) for 4 min at 95° C. for initial denaturation followed by 40 cycles at 95° C. for 30 sec, at 60° C. for 30 sec and at 72° C. for 90 sec. The final extension step was at 72° C. for 8 min.

TABLE 4
Correlation between the genotypes at 5′UTR-25 of RPH3AL and patient characteristics

| | Prospective sample (n = 95) | | | Retrospective sample (n = 134) | | | Combined sample (229) | | | |
|---|---|---|---|---|---|---|---|---|---|---|
| | Genotype | | | Genotype | | | Genotype | | | |
| | A/A | C/A | C/C | A/A | C/A | C/C | A/A | C/A | C/C | |
| | n = 9 | n = 27 | n = 59 | n = 11 | n = 48 | n = 75 | n = 20 | n = 75 | n = 134 | χ² |
| Characteristic | N (%) | N (%) | N (%) | N (%) | N (%) | N (%) | N (%) | N (%) | N (%) | P-value |

Age Group (years)

| | | | | | | | | | | |
|---|---|---|---|---|---|---|---|---|---|---|
| <65 | 5 (56) | 10 (37) | 24 (41) | 4 (36) | 20 (42) | 33 (44) | 9 (45) | 30 (40) | 57 (43) | 0.90 |
| ≧65 | 4 (44) | 17 (63) | 35 (59) | 7 (64) | 28 (58) | 42 (56) | 11 (55) | 45 (60) | 77 (57) | |

Gender

| | | | | | | | | | | |
|---|---|---|---|---|---|---|---|---|---|---|
| Female | 3 (33) | 9 (33) | 31 (53) | 3 (27) | 19 (40) | 45 (60) | 6 (30) | 28 (37) | 76 (57) | 0.006 |
| Male | 6 (67) | 18 (67) | 28 (47) | 8 (73) | 29 (60) | 30 (40) | 14 (70) | 47 (63) | 58 (43) | |

Ethnicity

| | | | | | | | | | | |
|---|---|---|---|---|---|---|---|---|---|---|
| African- | 0 (0) | 6 (22) | 33 (56) | 0 (0) | 9 (19) | 49 (65) | 0 (0) | 15 (20) | 82 (61) | <0.0001* |
| American | | | | | | | | | | |
| Caucasian | 9 (100) | 21 (78) | 26 (44) | 11 (100) | 39 (81) | 26 (35) | 20 (100) | 60 (80) | 52 (39) | |

Depth of tumor invasion

| | | | | | | | | | | |
|---|---|---|---|---|---|---|---|---|---|---|
| T₁ | 0 (0) | 4 (15) | 0 (0) | 0 (0) | 1 (2) | 1 (1) | 0 (0) | 5 (7) | 1 (1) | 0.01* |
| T₂ | 0 (0) | 6 (22) | 2 (3) | 2 (18) | 13 (27) | 14 (19) | 2 (10) | 19 (25) | 16 (12) | |
| T₃ | 6 (67) | 15 (56) | 47 (81) | 5 (46) | 25 (52) | 42 (56) | 11 (55) | 40 (53) | 89 (67) | |
| T₄ | 3 (33) | 2 (7) | 9 (16) | 4 (36) | 9 (19) | 18 (24) | 7 (35) | 11 (15) | 27 (20) | |

Nodal Status

| | | | | | | | | | | |
|---|---|---|---|---|---|---|---|---|---|---|
| Negative (N₀) | 0 (0) | 19 (70) | 28 (48) | 1 (9) | 33 (69) | 37 (49) | 1 (5) | 52 (69) | 65 (49) | <0.0001 |
| Positive (N₁₋₃) | 9 (100) | 8 (30) | 30 (52) | 10 (91) | 15 (31) | 38 (51) | 19 (95) | 23 (31) | 68 (51) | |

Distant metastasis

| | | | | | | | | | | |
|---|---|---|---|---|---|---|---|---|---|---|
| M₀ | 8 (89) | 23 (85) | 48 (83) | 6 (55) | 43 (90) | 62 (83) | 14 (70) | 66 (88) | 110 (83) | 0.15 |
| M₁ | 1 (11) | 4 (15) | 10 (17) | 5 (45) | 5 (10) | 13 (17) | 6 (30) | 9 (12) | 23 (17) | |

Tumor Stage^{†}

| | | | | | | | | | | |
|---|---|---|---|---|---|---|---|---|---|---|
| I | 0 (0) | 8 (30) | 1 (2) | 0 (0) | 12 (25) | 9 (12) | 0 (0) | 20 (27) | 10 (8) | <0.0001 |
| II | 0 (0) | 10 (37) | 22 (38) | 1 (9) | 20 (42) | 26 (35) | 1 (5) | 30 (40) | 48 (36) | |
| III | 8 (89) | 5 (18) | 25 (43) | 5 (46) | 11 (23) | 27 (36) | 13 (65) | 16 (21) | 52 (39) | |
| IV | 1 (11) | 4 (15) | 10 (17) | 5 (46) | 5 (10) | 13 (17) | 6 (30) | 9 (12) | 23 (17) | |

Tumor Location^{†}

| | | | | | | | | | | |
|---|---|---|---|---|---|---|---|---|---|---|
| Proximal Colon | 5 (66) | 9 (35) | 39 (66) | 4 (36) | 26 (54) | 31 (41) | 9 (45) | 35 (47) | 70 (52) | 0.71 |
| Distal | 4 (44) | 17 (65) | 20 (34) | 7 (64) | 22 (46) | 44 (59) | 11 (55) | 39 (53) | 60 (48) | |
| Colorectum | | | | | | | | | | |

Tumor Grade^{‡}

| | | | | | | | | | | |
|---|---|---|---|---|---|---|---|---|---|---|
| Low | 6 (75) | 19 (86) | 36 (67) | 8 (73) | 30 (63) | 44 (59) | 14 (74) | 49 (69) | 80 (62) | 0.44 |
| High | 2 (25) | 3 (14) | 18 (33) | 3 (27) | 18 (37) | 31 (41) | 5 (26) | 22 (31) | 49 (38) | |

Tumor Size (cms)^{‡}

| | | | | | | | | | | |
|---|---|---|---|---|---|---|---|---|---|---|
| ≦5 | 2 (22) | 16 (62) | 25 (47) | 4 (40) | 29 (62) | 40 (53) | 6 (32) | 45 (61) | 65 (51) | 0.06 |
| >5 | 7 (78) | 10 (38) | 28 (53) | 6 (60) | 18 (38) | 35 (47) | 13 (68) | 29 (39) | 63 (49) | |

*Fisher Exact Test; Data for tumor size and grade were not available in 100% of cases.
^{†}Data for tumor stage and location for one case was not available
^{‡}Data for tumor grade and size were not available in 100% of cases.

TABLE 5
Multivariate Cox proportional hazard analyses for survival
and risk of recurrence at five years after surgery.

| | | Recurrence | | | Survival | | |
|---|---|---|---|---|---|---|---|
| | | | 95% C.I.^{†} | | | 95% C.I.^{†} | |
| | | Hazard | (Lower- | p- | Hazard | (Lower- | p- |
| | | Ratio* | Upper) | value | Ratio* | Upper) | value |
| RPH3AL | C/C vs. C/A | 4.64 | 1.65-13.07 | 0.004 | 1.79 | 0.85-3.78 | 0.12 |
| Genotype | A/A vs. C/A | 13.85 | 3.12-16.43 | <0.001 | 3.88 | 1.39-10.82 | <0.01 |
| | A/A vs. C/C | 2.99 | 0.76-11.75 | 0.12 | 2.16 | 0.81-5.79 | 0.12 |
| Nodal | Pos.^{‡} vs. Neg.^{§} | 2.41 | 1.12-5.18 | 0.02 | 2.25 | 1.30-3.92 | <0.01 |
| Metastasis | | | | | | | |
| Tumor | High vs. Low | 1.27 | 0.61-2.68 | 0.62 | 1.95 | 1.14-3.31 | 0.01 |
| Grade | | | | | | | |
| Ethnicity | A-A^{¶} vs. | 0.89 | 0.41-1.95 | 0.77 | 1.77 | 0.97-3.24 | 0.06 |
| | Caucasians | | | | | | |
| Gender | Men vs. Women | 1.98 | 0.96-4.07 | 0.06 | 0.87 | 0.51-1.48 | 0.62 |

*Adjusted for the genotype of 5′UTR-25 of the RPH3AL gene, age, gender, and ethnicity, and nodal metastasis, depth of tumor grade, tumor location, and tumor size.
^{†}C.I. = Confidence intervals
^{‡}Pos. = Positive
^{§}Neg. = Negative
^{¶}A-A = African-Americans

Purification and Direct Sequencing of RPH3AL and p53

The PCR product was purified by enzymatic ‘PCR product pre-sequencing kit’ (USB Corporation, USA) after mixing with 1 μl (10 units) of exonuclease1 and 1 μl (2 units) of Shrimp Alkaline Phosphatase for each 5 μl of PCR amplification mixture, following incubation on Robocycler at 37° C. for 15 min to remove excess primers and dNTPs subsequently incubated at 80° C. for 15 min to inactivate enzymes. Purified PCR product was directly sequenced on ABI 3100 sequence detector. Compilation and sequence analysis was performed using the LASERGENE (DNA Star) sequence analysis software, which allows for direct analysis of sequencing electrophoretograms for the detection of duplex sequence signals at each position.

It was observed that all 6 CRCs which exhibited missense point mutations in the RPH3AL gene also exhibited missense point mutations in the p53 gene. The coexistence of missense point mutations in RPH3AL gene and p53 gene was statistically significant (Tables 2 and 6).

### Example 3

### DNA Mismatch Repair Deficiency and Microsatellite (Tandem DNA Sequence Repeats) Instability (MSI)

DNA mismatch repair deficiency and microsatellite (tandem DNA sequence repeats) instability (MSI) are hall marks of hereditary non-polyposis colorectal cancer [Boland, C. R., et al., A National Cancer Institute Workshop on Microsatellite Instability for cancer detection and familial predisposition: development of international criteria for the determination of microsatellite instability in colorectal cancer. Cancer Res, 1998.58: p. 5248-57] and a subset of sporadic CRCs [Chao, A., et al., Patient and tumor characteristics of colon cancers with microsatellite instability: a population-based study. Cancer Epidemiol Biomarkers Prev, 2000. 9(6): p. 539-44.; Slattery, M. L., et al., Associations between cigarette smoking, lifestyle factors, and microsatellite instability in colon tumors. J Natl Cancer Inst, 2000. 92(22): p. 1831-6]. The majority of sporadic CRCs (70%-80%) show LOH of 17p with no MSI (microsatellite stable). Several studies have reported an inverse correlation between MSI and p53 tumor-suppressor gene mutations in CRCs and suggested that mismatch repair deficiency provides a p53 independent pathway for development of CRC [Cottu, P. H., et al., Inverse correlation between RER+status and p53 mutation in colorectal cancer cell lines. Oncogene, 1996. 13(12): p. 2727-30]. To understand whether such correlation exist between the RPH3AL mutational status and MSI status, the MSI status was analyzed in these cases which were analyzed for RPH3AL mutations; however, no association between these two genetic events was observed. (Table 6).

Survival Analysis

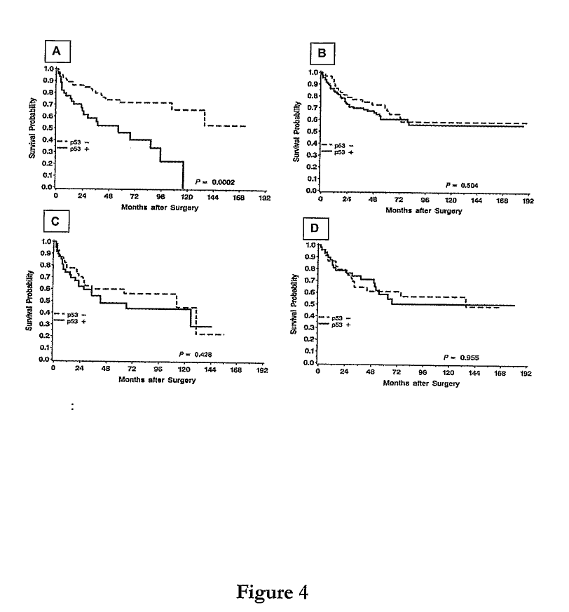

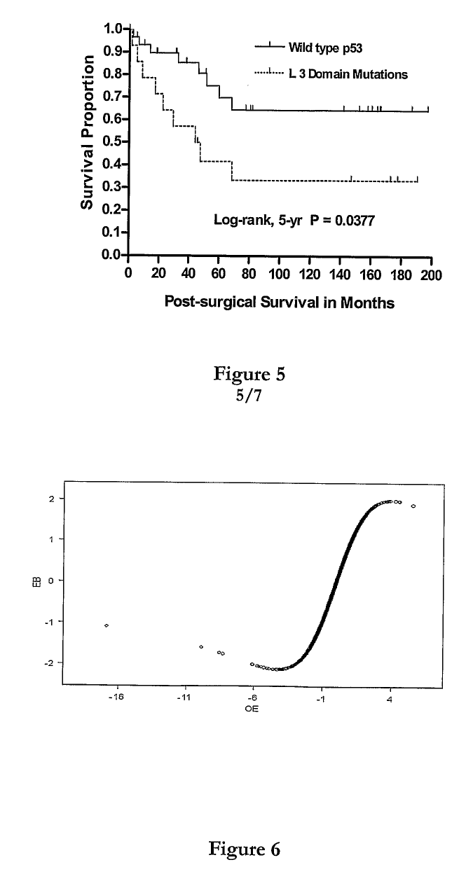

Previous studies of prognostic value of p53nac, detected by IHC, in well characterized CRC patient populations of African-Americans (n=204) and Caucasians (n=300) have demonstrated in univariate Kaplan-Meier survival analyses that Caucasian patients with CRCs that were located in the proximal colon and positive for p53nac had a poor overall survival (log rank, P=0.0002). However, such an association was not observed in distal tumors. In African-Americans, the p53nac status was not associated with overall survival of patients either with proximal or with distal tumors (FIG. 4) [Manne, U., et al., Nuclear accumulation of p53 in colorectal adenocarcinoma: prognostic importance differs with race and location of the tumor. Cancer, 1998. 83: p. 2456-67.]. Further analyses of the p53 gene mutations in proximal colon adenocarcinomas collected from African-American and Caucasian patients demonstrate a differential mutational pattern between African-American and Caucasian patients. The analysis indicated the majority of proximal tumors from African-Americans exhibited a wild-type p53 gene (72%), a small proportion of tumors exhibited point mutations (11%), and the rest 17% of CRCs had a base change at codon 72. The change at codon 72 with arginine (CGC) changed to proline (CCC) may be functionally equivalent [Matlashewski, G. J., et al., Primary structure polymorphism at amino acid residue 72 of human p53. Molecular & Cellular Biology, 1987. 7(2): p. 961-3] and this change has been described as an allelic polymorphic site in the p53 gene in several cancers including colorectal neoplasia, which varies with race [Matlashewski, G. J., et al., Primary structure polymorphism at amino acid residue 72 of human p53. Molecular & Cellular Biology, 1987. 7(2): p. 961-3; Beckman, G., et al., Is p53 polymorphism maintained by natural selection? Human Heredity, 1994. 44(5): p. 266-70]. In contrast, 62% of proximal tumors from Caucasians exhibited point mutations and interestingly all of them were point mutations and were located at ‘hot spot’ codons (175, 245, 248, 249, and 273) and a considerable number of cases (32%) had mutations in L3 domain of p53 (codons 245, 248 and 249). None of the proximal tumors from Caucasians exhibited changes at the polymorphic site codon 72. It is interesting to note that none of the CRCs from African-Americans exhibited mutations in L3 domain of the p53; whereas, polymorphisms at codon 72 (Arg>Pro) are more common in African-American patients as compared to Caucasians. Upon performance of Kaplan-Meier survival analyses between the Caucasian patient group of wtp53 and the patient group with L3 domain mutations and it was observed a statistically significant difference between these two groups in their survival at 5 years of post-surgery period (logrank, p=0.0377, FIG. 5).

Immunophenotypic Expression of the Cell Cycle Antigens

The mutational pattern of the RPH3AL gene in CRCs can also be determined. In addition, the phenotypic expression of p53, p21waf-1, p27kip-1, cyclin E, Ki67, Bax, Bcl-2, and the apoptotic index in the tissue specimens which were analyzed for RPH3AL gene status can be determined, because p53, p21waf-1, p27kip-1 and cyclin E are the key molecule in the regulation of cell cycle. The assessments of Ki67, and Bax, Bcl-2, expression are important to identify the rate of cell proliferation and apoptosis, respectively, in CRCs with inactivating (missense mutations) or partially functional mutations (SNPs) of the RPH3AL gene, since a considerable number of RPH3AL mutations reported in CRCs are SNPs. The mutational status of RPH3AL and phenotypic expression profiles of above mentioned molecules can also be assessed utilizing RT-PCR and direct DNA sequencing methods and immunohistochemistry assays, respectively. Again, appropriate statistical survival analyses on data sets generated can be assessed using univariate Kaplan-Meier and multivariate Cox regression analyses.

IHC Detection of Cell Cycle Antigens p53, p21^{waf-1}, p27^{kip-1} and Apoptosis

Phenotypic expression of p21waf-1, p27kip-1, and nuclear accumulation of p53 was also studied. Strong nuclear accumulation of p53 was noted in CRCs which exhibited a missense point mutations at codon 245. In addition, a strong nuclear immunostaining of p21waf-1 in CRCs wild-type for p53 was observed; whereas, the immunostaining pattern of cdk inhibitor, p27kip-1 was observed both in the cytoplasm and the nucleus in CRCs.

Apoptotic Index

Using a TUNEL assay, a higher apoptotic index was observed in adenomatous components of CRCs as compared to invasive CRCs. Specifically, a strong cytoplasmic IHC staining of Bax in CRC was observed. Also dual staining for Bax and apoptosis by IHC and TUNEL methods revealed Bax staining in the cytoplasm as well as apoptosis in the sample. Dual staining of BCL-2 (no staining in the cytoplasm) and apoptosis (staining in the cytoplasm) was observed in a serial tissue section.

TABLE 6
Correlation of RPH3AL mutational status with mutational
status of p53 and Microsatellite Instability status.

| | p53 gene status | | | | | MSI* | | |
|---|---|---|---|---|---|---|---|---|
| | Wt | | Mut | SNP | | MSS | MSI-H | |
| Type of RPH3AL | (N = 48) | | (N = 34) | (N = 13) | χ² | (N = 68) | (N = 14) | χ² |
| Mutation | N (%) | | N (%) | N (%) | P-value | N (%) | N (%) | P-value |
| Wild Type | 21 | (44) | 15 (44) | 10 (77) | | 29 (43) | 10 (71) | |
| Translatable Polymorphisms | 5 | (10) | 2 (6) | 0 (0) | 0.48 | 6 (8) | 0 (0) | 0.18 |
| Missense Mutations | 0 | (0) | 6 (18) | 0 (0) | <0.01 | 5 (8) | 0 (0) | 0.17 |
| Total Translatable Alterations | 5 | (6) | 8 (24) | 0 (0) | 0.10 | 11 (16) | 0 (0) | 0.10 |
| UTR-25 Genotype | | | | | | | | |
| C/C | 26 | (54) | 23 (67) | 10 (77) | 0.18 | 40 (59) | 10 (71) | 0.34 |
| C/A | 18 | (38) | 6 (18) | 3 (23) | | 19 (28) | 4 (29) | |
| A/A | 4 | (8) | 5 (15) | 0 (0) | | 9 (14) | 0 (0) | |

*= MSI status not available on 13 cases;
χ² = Chi-Square;
Wt = Wild Type;
Mut = Mutations;
SNP = Single Nucleotide Polymorphism (at codon 72) of p53;
MSS = Microsatellite Stable;
MSI-H = Microsatellite Instable;
UTR = Untranslatable Region;

### Example 4

### Gene Expression Studies on Human Xenografts Using Custom Made Gene Arrays

Gene Arrays

Gene expression studies were carried out on human prostate xenografts using custom-made gene arrays. The custom-made expression array contained 96 key apoptotic genes, 96 key cell cycle regulation genes and 75 stress and toxicity genes. Included in the custom arrays are genes involved in stress and toxicity, because the stress response may influence cell cycle regulation, cell differentiation, cell survival and apoptosis. These custom made microarrays were developed by Affimetrix.

Microarray Experimental Analysis

There is a wide variety of GeneChips available from Affymetrix for analysis of gene expression in humans. The Affymetrix custom array design program enables the design of custom expression arrays; thus, for these studies the use of custom made human apoptosis and cell-cycle gene arrays was made. This custom expression array will contain 96 key apoptotic genes, 96 key cell cycle regulation genes and 75 stress and toxicity genes (total number of genes per chip=267). In these custom arrays the genes included were involved in stress and toxicity, because stress response may influence cell cycle regulation, cell differentiation, cell survival and apoptosis. These custom made microarrays will be developed by several commercial companies.

The gene expression profiles of LS180, WiDr, and PC3 cell lines using custom made microarrays containing genes involved in cell cycle, proliferation and apoptosis was evaluated. An aliquot of the total RNA extracted was used. The RNA was used for the synthesis of cDNAs following standard protocols provided by the vendor. The transcriptional activity of a gene is determined by hybridizing fluorescently labeled first strand cDNAs corresponding to the experimental or control RNA sample to a microarray. The hybridization signal for each gene spotted on the array is determined using a laser confocal scanner. The intensity of the hybridization signal is representative of the expression level for the gene corresponding to that spot. The ratio of CY3 to CY5 signal is indicative of the change in gene expression between the two samples being analyzed.

Statistical Analysis of Microarray Data

The resulting data on the gene expression patterns and expression levels can be evaluated to identify the differences between control and the experimental settings; subsequently, differentially expressed gene profiles can be compared among different categories based on the genotype categories RPH3AL at −25 status using microarray specific statistical methods.

Data analysis includes from examination of data quality, normalization of intensity, gene selection, to clustering. For quality control, array comparability was checked by using intensity to examine replicate array variation prior to any data analysis. For normalization process, normalization methods, such as lowess smooth function and ANOVA model can be used for bias correction. For gene selection, approaches used in probe level data, such as, Dchip, MAS, RMA, percentile-range approach and the approaches, to identify differential expressed genes can be employed [Li, C. and W. Hung Wong, Model-based analysis of oligonucleotide arrays: model validation, design issues and standard error application. Genome Biol, 2001. 2(8); Chen, D. T., S. H. Lin, and S. J. Soong, Gene selection for oligonucleotide array: an approach using PM probe level data. Bioinformatics, 2004. 20(6): p. 854-62; Chen, D., A Graphical Approach for Quality Control of Oligonucleotide Array Data. Journal of Biopharmaceutical Statistics, 2004. 14: p. 591-606; Irizarry, R. A., et al., Summaries of Affymetrix GeneChip probe level data. Nucleic Acids Res, 2003. 31(4): p. 15]. The significant level from these statistical analyses can be evaluated by false-discovery rate or modified p value by permutation test. For clustering, the selected genes can be analyzed by various clustering methods, such as hierarchical clustering, self-organizing maps, and neural networks. Since different clustering approach may give different results, the common part for future investigation of pathway can be found.

Data Analysis

The microarray data on gene expression measurements of 267 genes, present in a custom made array, in 32 male nude mice xenografts was obtained and examined for the differences in gene expression levels. The data was analyzed using different approaches [Allison, D. B., Gadbury, G., Heo, M., Fernandez, J., Lee, C-K., Prolla, T. A., & Weindruch, R., A Mixture Model Approach For The Analysis of Microarray Gene Expression Data. Computational Statistics & Data Analysis, 2002. 39: p. 1-20]. For each gene, the ordinary and the empirical bayes (EB) estimate of the standardized difference was determined. Three different types of p-values were obtained: simple t-test p-values for the original data and log-transformed data both assuming equal variances, and chebby checker p-values. These p-values are presented on an individual basis as well as by taking multiple comparisons into account. The mix-o-matic method is applied to provide additional information about these p-values. The results shown here are the comparisons between control group and the 1 week castration group.

Results

The relationship between the empirical bayes (EB) and the ordinary estimate (OE) of the standardized difference is shown in FIG. 6. The relationship between the EB and OE estimate appears to be non-monotonic, which is theoretically undesirable. This non-monotonic relationship is possibly caused by the estimated standard error of the OE estimate, which heavily depends on the value of the corresponding OE estimate. Due to this standard error, relatively high values of the OE estimate will shrink the corresponding EB estimate too strongly towards the weighted grand mean over all OE estimates. Aim of this statistical analysis is to find which genes are significantly up regulated or significantly down regulated when control group was compared with the 1 week castration group. This up or down regulation of genes is examined by means of p-values for the null-hypothesis that there is no differential expression between groups and estimates of the standardized difference in these two groups. If for a particular gene this p-value is significant at the 5% level (p-value ≦0.05), then this gene is declared to be up-regulated when the estimated standardized difference is positive and declared to be down-regulated when this estimated is negative. If this p-value is non-significant (p-value >0.05) then this gene is declared to be not influenced. In addition to these initial data processing steps (FIG. 6) and utilization of additional analyses described in the sections D2.4 and D5.2, 29.5% of the genes observed differentially expressed in controls versus 1 week castrated groups.

### Example 5

### Assessment of p53, p21^{waf-1}, p27^{kip-1}, Cyclin E, Bax, and Bcl-2 Expression, and Proliferative (Ki67) and Apoptotic Index

The IHC expression of p53, p21waf-1, p27kip-1, cyclin E, Bax, and Bcl-2 to assess the cellular functions like cell growth, proliferation and the extent of apoptosis can be determined in CRCs evaluated for the RPH3AL status, as described below.

Immunohistochemistry

The phenotypic expression of p53, p21waf-1, p27kip-1, cyclin E, Bax, and Bcl-2 can be determined by IHC. Each of these are directly associated with different cellular pathways. The IHC techniques to be used have been described in detail several publications [Manne, U., et al., Prognostic significance of p27kip-1 expression in colorectal adenocarcinomas is associated with tumor stage. Clinical Cancer Research, Clinical Cancer Res 10:1743-1752, 2004; Manne, U., et al., Nuclear accumulation of p53 in colorectal adenocarcinoma: prognostic importance differs with race and location of the tumor. Cancer, 1998. 83: p. 2456-67; Manne, U., H. L. Weiss, and W. E. Grizzle, Bcl-2 expression is associated with improved prognosis in patients with distal colorectal adenocarcinomas. Int J Cancer, 2000. 89(5): p. 423-30; Manne, U., et al., Prognostic significance of Bcl-2 expression and p53 nuclear accumulation in colorectal adenocarcinoma. International Journal of Cancer, 1997. 74(3): p. 346-58; Manne, U., H. L. Weiss, and W. E. Grizzle, Racial differences in the prognostic usefulness of MUC1 and MUC2 in colorectal adenocarcinomas. Clin Cancer Res, 2000. 6(10): p. 4017-25; Manne, U., et al., Altered subcellular localization of suppressin, a novel inhibitor of cell-cycle entry, is an independent prognostic factor in colorectal adenocarcinomas. Clin Cancer Res, 2001. 7(11): p. 3495-503]. This procedure is based upon the use of a sandwich technique where primary antibody is biotin linked against secondary immunoglobulins. Detection is accomplished through an avidin-horseradish peroxidase complex together with the chromogen diamino-benzidine (DAB). The antibody suppliers, clones, working concentrations/dilutions and use of the antigen retrieval methods are shown in Table 7.

TABLE 7
List of Antibodies and IHC techniques

| | | IHC | IHC | ANTIGEN | STAINING |
|---|---|---|---|---|---|
| ANTIBODY | COMPANY | CLONE | DILUTION | RETRIEVAL | PATTERN |
| p53 | BioGenex | BP 53 | 1:80 | No | Nuclear |
| Ki67 | BioGenex | Mib-1 | 1:40 | Citrate Buffer | Nuclear |
| p21^{Waf-1} | Oncogene | EA10 | 1:20 | Citrate Buffer | Nuclear |
| p27^{kip-1} | NovoCastra | IB4 | 1:30 | Citrate Buffer | Nucl/Cyto |
| Cycline E | Santa Cruz | m-20 poly | 1:150 | To be evaluated | Nuclear |
| Bax | Santa Cruz | B-9 | 1:60 | Citrate Buffer | Cytoplasmic |
| Bcl-2 | Boehringer | 124 | 1:80 | Citrate Buffer | Cytoplasmic |
| | Mannheim | | | | |
| Apoptosis | R & D | CFZ31 | NA | TUNEL | Nuclear |
| | Systems | | | | |

All antigens, except p53 require a citrate-buffer based Microwave heating antigen retrieval (AR) technique. The AR procedure is not required for p53nac because of potential problems reported in CRCs [Manne, U., et al., Prognostic significance of Bcl-2 expression and p53 nuclear accumulation in colorectal adenocarcinoma. International Journal of Cancer, 1997. 74(3): p. 346-58; Baas, I. O., et al., Potential false-positive results with antigen enhancement for immunohistochemistry of the p53 gene product in colorectal neoplasms. Journal of Pathology, 1996. 178(3): p. 264-7]. As reported recently, the majority of antigens will be stable in the paraffin blocks [Manne, U., et al., Loss of tumor marker-immunostaining intensity on stored paraffin slides of breast cancer. J Natl Cancer Inst, 1997. 89: p. 585-6]. Known positive and negative control slides can be included in each staining run. Once the IHC is performed, the expression of different biomarkers can be analyzed as described below. Furthermore, a BLISS system or an updated CAS 200 system can be used.

Data Analysis

Phenotypic expression of p53nac, p21waf-1, p27kip-1, cyclin E, Ki67, Bax, and Bcl-2, as detected by IHC, can be evaluated on tissue sections from tissue blocks prepared from CRCs analyzed for RPH3AL mutational status. Data from all the patients can be correlated with the mutational status of RPH3AL to the aggressive nature of the RPH3AL mutations.

As a control, several cell lines that express proteins at known intensities and sub-cellular locations (e.g. WiDr for mutant p53, LS180 for wild-type p53 (no staining), SKOV3 for erbB-2 etc.) can be used and cell blocks can be made from these cell lines after fixing in formalin. The sections from these cell blocks can be stained along with the CRC tissues sections to create a “standard curve” after quantification using the image analysis instrumentation.

Staining Evaluations

To limit the bias and to resolve differences in interpretations of staining, at least two independent evaluations of the assessment of phenotypic expression can be employed. First, each biomarker can be analyzed in the uninvolved mucosa and then in the invasive adenocarcinoma. The evaluation of nuclear markers can then be assessed in at least 500 cells using a X40 objective. To evaluate these nuclear antigens, both percent positivity and staining intensity can be assessed separately; whereas, a semi-quantitative immunostaining score (ISS) can be estimated for cytoplasmic expression. In brief, the intensity of staining of individual cells can be scored on a scale of 0 to +4. In addition, each evaluation can generate an estimate of the proportion of cells stained at each intensity level. The percent of cells and the corresponding intensity can then be multiplied to obtain the ISS. The immunostaining scores of all evaluations can be combined to obtain the mean ISS [Manne, U., et al., Nuclear accumulation of p53 in colorectal adenocarcinoma: prognostic importance differs with race and location of the tumor. Cancer, 1998. 83: p. 2456-67; Manne, U., et al., Prognostic significance of Bcl-2 expression and p53 nuclear accumulation in colorectal adenocarcinoma. International Journal of Cancer, 1997. 74(3): p. 346-58; Manne, U., H. L. Weiss, and W. E. Grizzle, Racial differences in the prognostic usefulness of MUC1 and MUC2 in colorectal adenocarcinomas. Clin Cancer Res, 2000. 6(10): p. 4017-25.; Manne, U., et al., Altered subcellular localization of suppressin, a novel inhibitor of cell-cycle entry, is an independent prognostic factor in colorectal adenocarcinomas. Clin Cancer Res, 2001. 7(11): p. 3495-503]. The staining patterns of the markers are shown in Table 7.

In Situ Detection of Apoptosis

The growth of a tumor is frequently reflective of the ratio of cell proliferation to apoptosis. An alternate to the “DNA ladder” method for examining apoptosis via DNA fragmentation, is by use of the TUNEL assay. However, DNA fragmentation is a characteristic of both apoptotic as well as necrotic cells. To discriminate apoptotic cells from necrotic cells, an additional method of apoptosis detection can be used. For example, immunoreactivity of active caspases within a cell in addition to TUNEL staining can indicate that cell is apoptotic but not necrotic. The rate of apoptosis can be measured in CRCs evaluated for RPH3AL mutational status using a newly developed in situ apoptosis detection kit, TUNEL/caspase-3 double labeling assay (R&D Systems, MN). The TUNEL/casepase-3 double-labeled cells demonstrate with both dark-brown nuclei and red-stained cytoplasm. The percent positive cells with distinct nuclear as well as cytoplasmic staining can be evaluated to assess the extent of apoptosis. The apoptotic index can be correlated with RPH3AL mutations as well as with other phenotypic markers.

Data Evaluation

The data of these studies can be analyzed to determine the effects of RPH3AL mutations on the growth, proliferation and apoptosis. The type of RPH3AL mutations can be correlated with the rate of cell growth and proliferation, and the extent of apoptotic cell death.

The statistical analysis methods and strategies to correlate the data of molecular expression detected by IHC, with different factors and with patient survival can be performed as described below.

Statistical Analyses

The differences in proportions of point mutations and polymorphisms can be tested for statistical significance using Pearson's Chi-square or Fisher's exact test as appropriate. Indicator variables can be created to identify individuals with and without the mutations. These variables can be used as stratifiers in Kaplan Meier analysis to compare survival between the group with a mutation and the group without. Cox's proportional hazard regression model can be used to fit a multivariable model to the data to assess the significance of the mutations in predicting time to death (or recurrence) after controlling for other factors such as age, etc.

As noted above, analysis can include evaluating the predictive ability of mutations in different regions of RPH3AL (e.g. UTR-25 versus non-UTR-25 mutations or wild type vs. mutations in UTR-25). The outcome to be measured in these studies can be the survival. This outcome can be assessed first univariately, using Kaplan-Meier curves and stratified for the presence or absence of mutations in RPH3AL. To compare the survival rates between UTR-25 RPH3AL-mutant vs. RPH3AL-wt group of patients, the log-rank test of significance can be used. The mutation can be considered as a univariate predictor if the p-value for the log-rank test is <0.013 (p=0.01 bonferroni correction for five mutation sites). Median survival time, survival rates at specific time points along with 95% confidence intervals can be calculated in patients with tumors positive for mutation or marker expression versus those without mutation or with lack of or decreased expression.

Cox proportional hazards regression can be used to assess the predictive value of RPH3AL mutations in different domains, particularly alteration at UTR-25 of RPH3AL, after controlling for tumor stage, grade and its anatomic location within the colorectum. The P-values for tests of interactions can be computed by the use of a likelihood ratio statistics comparison model with only main effects. This can be compared to a model to identify significant predictors of treatment failure or patient survival. Hazard ratios and 95% confidence intervals can be calculated based on the significant variables in the model.

The predictive value of RPH3AL mutations together with other 7 molecular markers (p53 and others of this specific aim) can be assessed using Kaplan-Meier survival curves and a Cox proportional hazards regression model.

Recognizing the importance of tumor stage in patient survival, subgroup analyses according to the tumor stage can also be performed. The importance of each mutation category and tumor stage subgroup using Kaplan-Meier curves can also be assessed. Likewise, the log-rank test can be employed to determine any significant differences in survival curves, for example, in patients with Stage III tumors and whose CRCs exhibit SNP at UTR-25 in RPH3AL and patients without RPH3AL mutations.

Statistical Validation

The validity of the findings can be assessed to learn whether the results are reproducible. The data as well as the statistical models can be validated using “bootstrapping” [Efron, B. a. T., R J, An introduction to the bootstrap. 1993, London, UK: Chapman & Hall], “leave-one-out” cross-validation [Feinstein, A., Multivariable analysis: An introduction. 1996: Yale University Press, New Haven, 1996] or “split-sample validation” [Harrell, F., Regression modeling strategies: With applications to linear models, logistic regression, and survival analysis. 2001: Springer-Verlag, New York] as appropriate. These methods are used for estimating generalization errors which are the basis for choosing among various models. Bootstrapping is a way of testing the reliability of the dataset. It is the creation of pseudoreplicate datasets by resampling. Bootstrapping allows for assessment of whether the distribution of characters has been influenced by stochastic effects. Bootstrapping works better than cross-validation in many cases [Efron, B., Estimating the error rate of a prediction rule: improvement on cross-validation. J. of the American Statistical Association, 1983. 78: p. 316-331]; however, leave-one-out cross-validation is markedly superior for small data sets. Further, the methodology proposed by Altman and Royston [Altman, D. G. and P. Royston, What do we mean by validating a prognostic model? Statistics in Medicine, 2000. 19(4): p. 453-73] can be used for validation of prognostic models. Although these methods are commonly used in classifier development, recently their usage in cancer molecular-marker discovery data was discussed [Ransohoff, D. F., Rules of evidence for cancer molecular-marker discovery and validation. Nat Rev Cancer, 2004. 4(4): p. 309-14]. The approach used can be to randomly select a percentage of the overall data, stratified on the binary dependent or target variable to ensure equivalence, to be a “training or fitting” data set, and a “validation or test” data set. Furthermore, data collection can be ongoing. The data collected during the later part of these studies can be used as a “independent validation or test” data set to obtain the unbiased estimates of our models' accuracy and to demonstrate reproducibility of any results. All these statistical validation methods not only address the problems related to overfitting, but also check for goodness-of-fit and assess the unbiasedness of the predictions on new data.

Addressing Missing Data Issues

To deal with missing data, a method of non-ignorable non-response (NINR) in a longitudinal data set that is fitted to a mixed model can be used. This method can be adapted to Cox's proportional hazards model to test the set of missing responses for NINR. if the missing data is shown to be ignorable, various techniques such as data augmentation, EM algorithms [Schafer, J., Analysis of incomplete multivariate data. London: Champman and Hall], and sensitivity analysis can be used on the parameter estimates. With respect to missing covariate data, multiple imputations [Schafer, J., Analysis of incomplete multivariate data. London: Champman and Hall] to fill in plausible values can be used. The sensitivity analysis for the parameter estimates can first look at the two techniques for filling in missing responses, then responses and covariates, and then just covariates.

### Example 6

### Identification of the Gene Expression Profiles of CRCs with or without Aggressive RPH3AL Mutations

Missense mutations in RPH3AL in CRCs which exhibited missense mutations in the p53 gene are shown (Table 6). See also [Goi, T., et al., Mutations of rabphillin-3A-like gene in colorectal cancers. Oncol Rep, 2002. 9(6): p. 1189-92]

To determine a possible function of RPH3AL the gene expression profiles of CRCs with aggressive, non-aggressive RPH3AL mutations and with wt-RPH3AL can be determined based on the findings described above, to identify the type and the levels of expression of different genes that are dependent on the p53 status. The identification of patterns of gene expression in CRCs from the patient groups described above can determine specific differences which may aid in understanding the genetic variations which contribute to the aggressive progression of CRCs.

Microarray Experimental Analysis:

A wide variety of GeneChips available from Affymetrix for analysis of gene expression in humans can be used. For example, a custom made human apoptosis and cell-cycle gene arrays can be used. This custom expression array can contain 96 key apoptotic genes, 96 key cell cycle regulation genes and 75 stress and toxicity genes (total number of genes per chip=267).

48 CRCs can be evaluated for their gene expression profiles. An aliquot of the total RNA extracted can be used. The RNA can be used from 8 CRCs and from 8 matching uninvolved epithelia (8 cms away from the tumor) for each of the three groups categorized based on the mutational status of RPH3AL. Standard protocol provided by the vendor can be used. Briefly, the transcriptional activity of a gene is determined by hybridizing fluorescently labeled first strand cDNAs corresponding to the experimental or control RNA sample to a microarray. The hybridization signal for each gene spotted on the array is determined using a laser confocal scanner. The intensity of the hybridization signal is representative of the expression level for the gene corresponding to that spot. The ratio of CY3 to CY5 signal is indicative of the change in gene expression between the two samples being analyzed. Gene chip analysis can then be conducted.

Statistical Analysis of Microarray Data.

Several novel methods can be used to analyze the data generated by the microarray. [Li, C. and W. Hung Wong, Model-based analysis of oligonucleotide arrays: model validation, design issues and standard error application. Genome Biol, 2001. 2(8): p. RESEARCH0032; www.iarc.fr/p53/index.html; Chen, D. T., S. H. Lin, and S. J. Soong, Gene selection for oligonucleotide array: an approach using PM probe level data. Bioinformatics, 2004. 20(6): p. 854-62]. The resulting data on the gene expression patterns and expression levels can be evaluated to identify the differences between normal and tumor tissues; subsequently, differentially expressed gene profiles can be compared among the tumor categories based on the RPH3AL status using microarray specific statistical methods.

Data analysis can include from examination of data quality, normalization of intensity, gene selection, to clustering. For quality control, array comparability can be performed by using intensity to examine replicate array variation prior to any data analysis [Chen, D., A Graphical Approach for Quality Control of Oligonucleotide Array Data. Journal of Biopharmaceutical Statistics, 2004. 14: p. 591-606]. For normalization process, normalization methods, such as lowess smooth function and ANOVA model can be used for bias correction. For gene selection, approaches used in probe level data, such as, Dchip, MAS, RMA, percentile-range approach can be employed as well as the approaches described elsewhere herein, to identify differential expressed genes [Li, C. and W. Hung Wong, Model-based analysis of oligonucleotide arrays: model validation, design issues and standard error application. Genome Biol, 2001. 2(8): p. RESEARCH0032; www.iarc.fr/p53/index.html.; Chen, D. T., S. H. Lin, and S. J. Soong, Gene selection for oligonucleotide array: an approach using PM probe level data. Bioinformatics, 2004. 20(6): p. 854-62; Irizarry, R. A., et al., Summaries of Affymetrix GeneChip probe level data. Nucleic Acids Res, 2003. 31(4): p. e15]. The significant level from these statistical analyses can be evaluated by false-discovery rate or modified p value by permutation test. For clustering, the selected genes can be analyzed by various clustering methods, such as hierarchical clustering, self-organizing maps, and neural networks.

Immunohistochemistry (IHC)

The abnormal phenotypic expression patterns of key molecular markers can also be evaluated using immunohistochemistry (IHC). Possible markers to be analyzed include, but are not limited to: The suppressor gene p53; the cell cycle antigens, p21 Waf-1, p27Kip-1, cyclin E; the proliferation marker, Ki67; and the apoptotic antigens, Bcl-2 and Bax.

Apoptosis Assay

In addition the extent of apoptosis can be determined utilizing TUNEL/caspase-3 double labeling assay. Apoptosis is responsible for the programmed death of cells with mutant DNA. The index of apoptosis is usually measured by a simple IHC method using the terminal deoxynucleotidyl transferase mediated digoxigenin nick end labeling technique (TUNEL). A higher number of apoptotic index was observed in colorectal adenomas as compared to invasive CRCs [Konopleva, M., et al., Apoptosis. Molecules and mechanisms. Advances in Experimental Medicine & Biology, 1999. 457: p. 217-36]. Several studies have demonstrated an association between changes in the apoptotic index and the progression of CRCs [Nomura, M., et al., Morphogenesis of nonpolypoid colorectal adenomas and early carcinomas assessed by cell proliferation and apoptosis. Virchows Archiv, 2000. 437(1): p. 17-24; Sinicrope, F. A., et al., Increased apoptosis accompanies neoplastic development in the human colorectum. Clinical Cancer Research, 1996. 2(12): p. 1999-2006].

### Example 7

### Gene Expression Data Analysis

The Affymetrix GeneChip software utilizes several algorithms to analyze the results from the GeneChip hybridization for the experimental and control samples. It then calculates a set of metrics that describe background signal and the behavior of each oligonucleotide (16-20 for each gene) correspond to a gene on the array. To compare the level of expression of a gene in the control and experimental samples, the overall hybridization signal on each array is standardized using the average intensity all the genes (˜10-12,000) on the array rather than a few select “housekeeping” genes.

The purpose of the Affymetrix MicroDB software is to create, manage, publish, and archive data derived from the analysis software. The databases generated by MicroDB are Genetic Analysis Technology Consortium (GATC) compliant and allow the expression data to be further evaluated using the Affymetrix Data Mining Tools (DMT) as well as any other third party GATC compliant expression analysis software. The Affymetrix DMT software filters data and allows multifaceted queries to be built for extracting the most meaningful and interesting results from the complex datasets. The filtered data can rapidly be sorted, grouped, and presented using several different visual formats such as scatter plots, bar graphs, pivot tables, hierarchical clusters and self organizing maps.

### Statistical and Data Mining Analyses to Predict Patient Prognosis.

Statistical Analyses.

Estimates of patient prognosis are important for diagnostic and therapeutic decision making and for selection of patients for randomized clinical trials. The estimates may be obtained by regression analysis with individual patient data [Harrell, F., K. Lee, and D. Mark, Multivariable prognosis models: issues in developing models, evaluating assumptions and adequacy, and measuring and reducing errors. Statistics in Medicine, 1996. 15: p. 361-87.]. For dichotomous outcomes, logistic regression models can be used to provide probability estimates in relation to patient characteristics. The larger study population is advantageous for prognostic modeling, since the regression coefficients can be estimated more accurately, and the statistical power increases. However, current statistical methods are cumbersome in handling the vast amount of data necessary for comprehensive evaluation and integration of molecular and clinicopathologic factors [Fielding, L. P. and D. E. Henson, Multiple prognostic factors and outcome analysis in patients with cancer. Communication from the American Joint Committee on Cancer. Cancer, 1993. 71(7): p. 2426-9; Simon, R. and D. G. Altman, Statistical aspects of prognostic factor studies in oncology. Br J Cancer, 1994. 69(6): p. 979-85]. Newer statistical tools including neural networks and decision trees, have been proposed as alternate solutions, but these require the availability of large data sets [Burke, H. B. and D. E. Henson, The American Joint Committee on Cancer. Criteria for prognostic factors and for an enhanced prognostic system. Cancer, 1993. 72(10): p. 3131-5].

Artificial Intelligence and Computational Data-Mining

The characterizing feature of data-mining techniques is their exploratory nature. Such techniques are already being proposed for developing models to detect and predict adverse events in patient safety applications. Data mining has been successfully applied to the problems of hospital infection control surveillance [Brossette, S. E., et al., Association rules and data mining in hospital infection control and public health surveillance. J Am Med Inform Assoc, 1998. 5(4): p. 373-81; Brossette, S. E., et al., A data mining system for infection control surveillance. Methods Inf Med, 2000. 39(4-5): p. 303-10; Moser, S. A., W. T. Jones, and S. E. Brossette, Application of data mining to intensive care unit microbiologic data. Emerg Infect Dis, 1999. 5(3): p. 454-7; McDonald, J. M., S. Brossette, and S. A. Moser, Pathology information systems: data mining leads to knowledge discovery. Arch Pathol Lab Med, 1998. 122(5): p. 409-11.; Brossette, S, and R. M. Wartell, A program for selecting DNA fragments to detect mutations by denaturing gel electrophoresis methods. Nucleic Acids Res, 1994. 22(20): p. 4321-5] as well as to cancer research [Manne, U. and e. al. Use of artificial intelligence (AI) and computational statistical (CS) methods in predicting the clinical outcome of colorectal cancer (CRC) patients. in Proc AACR, Vol. 41, Abstract No. 1534. 2001. New Orleans, La.; Liu, D., A. Sprague, and U. Manne, Dynamically constructing classification rules with visualization techniques. Int. J. Comp Sciences & Comp Eng, 2004. 6: p. 40-46.; Liu, D., A. Sprague, and U. Manne, JRV: An interactive tool for data mining visualization. J. ACMSE, 2004. 4: p. 442-447].

Data mining is a data driven descriptive exercise in which the goal is often pattern recognition [Hand, D. J., Data Mining: Statistics and More? American Statistician, 1998. 52: p. 112-118; Hand, D. J., Data Mining—Reaching Beyond Statistics. Res. Official. Statist., 1998. 2: p. 5-17; Hand, D. J., Statistics and Data Mining: Intersecting Disciples. SIGKDD Exploration, 1999. 1: p. 16-19; Hand, D. J., et al., Data Mining for Fun and Profit. Statistical Science, 2000. 15: p. 115-121]. The actual tools used in data mining are familiar to most statisticians; e.g., decision trees, logistic regression, and cluster analysis but the use of these tools has a different emphasis. Whereas a proportional hazards model can be of interest from the perspective of examining risk factors associated with a given disease such as colorectal cancer, a decision tree used in the framework of data mining may have as its focus the clinical stages through which a given patient moves over the course of their illness.

Recently Zhang et al. [Zhang, H., et al., Recursive partitioning for tumor classification with gene expression microarray data. Proc Natl Acad Sci USA, 2001. 98(12): p. 6730-5] utilized recursive partitioning to improve classification of tissues on the basis of gene expression data. Indeed, studies in lung [Cox, L. A., Jr., Does diesel exhaust cause human lung cancer? Risk Analysis, 1997. 17(6): p. 807-29] and colorectal [Bottaci, L., et al., Artificial neural networks applied to outcome prediction for colorectal cancer patients in separate institutions nts]. Lancet, 1997. 350(9076): p. 469-72] neoplasia have demonstrated distinct differences in interpretation of epidemiological risk assessment results obtained from complex multivariate data sets utilizing standard bio-statistical analyses and artificial intelligence and data-mining techniques.

### Example 8

### RPH3AL Gene Expression Based on the SNP at 5′UTR-25

To assess the functional importance of SNPs at 5′UTR-25, the mRNA levels of RPH3AL were quantified by quantitative real time PCR (QRT-PCR).

Quantitative RT-PCR Analysis

Total RNA was extracted directly from frozen tissues and matching normal tissues of prospective cases by use of an RNeasy Kit (QIAGEN) and 1 μg of total RNA was reverse transcribed. PCR was performed in SYBR green reagent supermix (Bio-Rad laboratories, Hercules, Calif.) in a final volume of 25 μL consisting of 0.5 μL of each primer (5 pmoles), 12.5 μL of 2× supermix containing the reaction buffer, Fast-start Tag DNA double strand-specified SYBR green I dye, 6.5 μL of nuclease free water and 5 μL of cDNA template following incubation of PCR reactions at 95° C. for an initial denaturation followed by 45 cycles of 15 sec denaturation at 95° C., and annealing and extension at 57° C. for 30 sec. The PCR reactions were performed using i-Cycler Real-time PCR system (Bio-Rad). The sequences of primers used for PCR analysis are as follows: RPH3AL, 5′-CGAGGATCGTCTGCCTTATT-3 (sense) SEQ ID NO: 19 AND 5′-GCACGTACAAGTGTCCACTACA-3′ (antisense) SEQ ID NO: 20 and β-Actin, 5′-TAAGTAGGCGCACAGTAGGTCTGA-3 (sense) SEQ ID NO: 21 and 5′-AAGTGCAAAGAACACGGCTAAG-3 (antisense) SEQ ID NO: 22. PCR products were subjected to melting curve analysis to exclude non-specific amplification. All of the PCR reactions were performed in sets of four. Mean of RPH3AL mRNA and β-Actin mRNA copy numbers were calculated for each patient separately and a ratio of mean RPH3AL mRNA and β-Actin mRNA.

Results

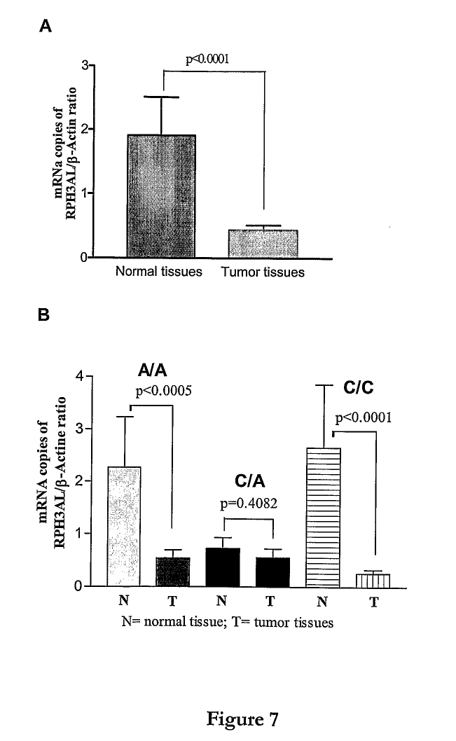

The PH3AL mRNA expression was significantly reduced in tumors compared to matching benign normal colonic epithelial tissues, and the difference between these two groups of tissues was significantly different (χ², p<0.0001) (FIG. 7A). Analysis of RPH3AL mRNA levels based on three genotypes of SNP at 5′UTR-25 of the RPH3AL gene suggested that there was a significant difference between the in tumors and in their matching normal tissues, specifically in the genotypes A/A and C/C, but not in C/A (FIG. 7B).

Although most polymorphisms are functionally neutral, some of them, like SNP at 5′UTR, may be affecting the regulation of the RPH3AL gene expression. The down regulation of intratumoral expression of RPH3AL gene compared to matching normal tissues suggests that RPH3AL gene is a candidate tumor suppressor gene of CRC and possibly in other sporadic carcinomas. These functional polymorphisms, despite being of low occurrence, could contribute to the differences between individuals in susceptibility and severity of disease.

### Example 9

### Linkage Disequilibrium Studies of the RPH3AL Gene

Polymorphisms in association with other genetic or epigenetic events or in strong linkage disequilibrium with other SNPs or loss of heterozygosity (LOH) are involved in pathogenesis of cancer through modulating gene expression (Kawakami K. et al. 2002). Also, LOH is a well known genetic event in various cancers which have profound effect on allele copy number change and level of gene expression of several tumor suppressor genes (Wieland I, 1999), and is involved in tumor progression, poor survival and early recurrence of several human malignancies (Shirasaki F, 2001). Therefore, LOH at RPH3AL locus and 13 additional SNPs in the genomic region of RPH3AL gene were evaluated to examine its effect on RPH3AL expression based on the genotype of SNP at 5′UTR-25.

Analysis of Loss of Heterozygosity

Genomic DNA extracted from CRCs and matching normal tissues was used to examine LOH status of RPH3AL locus. LOH at polymorphic loci D17S1866, D17S926, D17S643 and D17S849 was evaluated in CRC and normal specimens using primers deposited in the database of NCBI and are shown in Table 8. In each primer set, forward primer was labeled with fluorescent dye for allele detection (Applied Biosystems, Inc., Foster City, Calif.). The 25 μL reaction mixture consisted of 10×PCR buffer, 10 mM of each dNTP, 15 mM of MgCl₂, 10 pmoles of each primer, 0.3 μL (2.5 units) of Platinum Taq Polymerase (Invitrogen, Carlsbad, Calif.), and 100 ng of genomic DNA. Amplification was achieved by 2 min of initial denaturation at 95° C. followed by 35 cycles of 30 seconds at 95° C., 30 seconds at 60° C., 1 min at 72° C. and a 20 min final extension at 72° C. Two microlitres of each labeled PCR product was added to the mix of 12 μl deionized formamide and 1 μl of Gene Scan 500 ROX (Applied Biosystem) and then was denatured at 88° C. for 5 min followed by chilling on ice for 2 min and spin for 15 s. Microcapillary electrophoresis of PCR products was carried out in an ABI 3100 genetic analyzer (Applied Biosystem). The data were collected automatically and analyzed by Genotyper 2.1 software (Applied Biosystem) to interpret the data. Allelic loss was from informative microsatellite markers calculated according to a previously described formula (Liloglou, T., et al 2000 and 2001).

Results

In the present study 22 CRCs and corresponding normal tissues that were wild type for mutations and polymorphisms in the coding region of RPH3AL were analyzed to assess status of LOH, and mRNA expression. Thirteen CRCs (13 of 22, 59%) exhibited LOH at one or more of the four markers assessed. The highest frequency of LOH was found in CRCs which demonstrated the A/A (5 of 5,100%) or C/C (7 of 10, 70%) variant genotypes at 5′UTR-25 of RPH3AL gene, whereas the C/A variant genotype was observed only in one tumor (1 of 7, 14%) (Table 9). The same sample set was analyzed for the mRNA levels of RPH3AL gene to assess the relationship between LOH and its expression status, because chromosome 17p is a site of frequent deletions that have a substantial effect on copy number change and level of gene expression. The ratio of RPH3AL mRNA to β-Actin mRNA copy numbers was determined and correlated with the LOH status (Table 9). There was a significant association between RPH3AL expression and LOH. The levels of mRNA expression were significantly higher in normal (benign) tissues 2.36 (0.15 to 10.32) compared to matching invasive tumor tissues 0.42 (0.038 to 1.70). As shown in Table 9, significantly decreased mRNA expression of RPH3AL gene was observed in CRCs which exhibited A/A (0.38, range 0.06 to 0.59) or C/C (0.27, range 0.038 to 0.746) variant genotypes compared to the C/A variant genotype (0.659, range 0.58 to 1.78). However; there was no significant difference between CRC and the corresponding normal tissues of patients that are heterozygous for C/A variant genotype. The reduction of mRNA expression was higher in CRCs those with LOH (0.24, range 0.04 to 0.746) compared to those without LOH (0.54, range 0.038-1.78).

### Example 10

### Analysis of 13 SNPs Located in the Genomic Region of the RPH3AL Gene

The information on 13 SNPs were obtained from the dbSNP database of the NCBI (http://www.ncbi.nlm.nih.gov/SNP). Details of their IDs, the PCR primer sets and their position on the chromosome 17 are shown in Table 10. The following methods of PCR and sequencing were utilized. The 25 μL reaction mixture consisted of 10×PCR buffer, 10 mM of each dNTP, 15 mM of MgCl₂, 10 pmoles of each primer, 0.5 μL (2.5 units) of Platinum Taq Polymerase (Invitrogen), and 100 ng of genomic DNA. Amplification was achieved by 5 min of initial denaturation at 94° C. followed by 35 cycles of 30 seconds each at 94° C., 30 seconds each at 60° C., 1 min at 70° C. and a 7-min final extension at 70° C. PCR products were fractionated by electrophoresis in a 2% agarose gel and stained with ethidium bromide.

Results

The findings of analyses of the 13 SNPs were correlated with the genotype status of SNP at 5′UTR-25 of RPH3AL gene. As shown in Table 10, there was no significant association between the 5′UTR-25 genotypes (A/A, C/C and C/A) and SNPs in the genomic region of RPH3AL, indicating that 5′UTR-25 variants were not in linkage disequilibrium with other SNPs. Although, mutant alleles were observed in three SNPs (SNP Cluster IDs rs9907777, rs12942039 and rs12949751), they were evenly distributed among three genotype variants of SNP at 5′UTR-25 of RPH3AL (Table 10).

In agreement with several previous reports on expression of tumor suppressor genes and LOH (Wieland I, 1999, Shirasaki F, 2001, Kawakami K. et al. 2002), the present study has also observed the down regulation of RPH3AL expression in CRCs with LOH at 17p13.3 regardless of the status of several other SNPs or different variant genotypes of SNP at 5′UTR-25. The higher incidence of LOH observed in CRCs with A/A and C/C variant genotypes of SNP at 5′UTR-25, compared to C/A variant genotype, the LOH might be one of the possible genetic mechanisms responsible for down regulation of RPH3AL expression in CRCs, and contributing to the aggressive tumor behavior resulting in early disease recurrence and poor patient survival. In contrast, a lower incidence of LOH of the RPH3AL gene in CRCs with C/A heterozygote variants of SNP at 5′UTR-25 might be a possible contributor for better patient survival; however, the molecular mechanisms for this association remains unclear. Another possible explanation for better survival of patients whose CRCs exhibit C/A alleles at 5′UTR-25 SNP that the C/A heterozygotes may aid in providing adequate amounts of mRNA during translation process or in perfect protein structure formation. A tumor suppressor gene may also be inactivated by homozygous deletion, or promoter methylation or inactivation of transcription machinery that would not involve LOH. This may be one of the reasons why other tumors that did not exhibit LOH at 17p13.3 were low expressers of RPH3AL.

### Example 11

### Gene Expression Studies on CRC Samples Analyzed for the Status of the SNP at 5′UTR-25 of RPH3AL Gene

The purpose of these studies was to obtain a better understanding of cellular and molecular events of in CRCs with SNPs at the 5′UTR-25 of RPH3AL.

Using Affimetrix Gene Arrays

Gene expression analysis on human CRC samples were conducted using The Affymetrix Human Genome U133 Plus 2.0 array (Affymetrix, Santa Clara, Calif.) was used to study the gene expression profiles of tumors and the matching benign control colonic epithelial tissues. The array contains over 47,000 transcripts including 38,500 well known genes. The gene expression microarray studies were performed on 4 pairs of CRCs tissues (normal and tumor) from each genotype category (A/A, C/A and C/C) (in total 12 CRCs and 12 matching controls).

The specimens selected were non-Hispanic Caucasian patients who received only surgery as therapy without any pre- or post surgery chemo or radiation therapies. Standard protocols provided by the vendor (Affymetrix) were used. Briefly, aliquots of the total RNA extracted from prospectively collected frozen specimens (CRCs and matching uninvolved epithelial tissues 8 cms away from the tumor) were used. Five micrograms of total RNA from each specimen was submitted for gene expression analysis. The quality of the total RNA was determined using the RNA nanochip on an Agilent BioAnalyzer (Agilent Biotechnologies, Palo Alto, Calif.) before proceeding to synthesis of double strand cDNA. Double-stranded cDNA was generated by linear amplification using oligo dT-T7 primer and reverse transcriptase. Subsequently, biotin-labeled cRNA was synthesized by in vitro transcription (IVT) using the 3′-amplification reagents for IVT labeling (Affymetrix). After the quality of the cRNA was determined, cRNA was fragmented into 50-200 base fragments to ensure more uniform hybridization kinetics. Prior to hybridizing to the expression arrays, the quality of the hybridization target was determined by hybridization to a Test3 array that indicated the efficacy of the RT/IVT reaction by the ratios of expression level of 5′ to 3′ house-keeping genes (β-actin and GAPDH). The arrays were hybridized overnight at 45° C. for 16 hrs and washed, stained, and scanned the next day. The transcriptional activity of a gene was determined by hybridizing fluorescently labeled first strand cDNAs, corresponding to the experimental or control RNA sample, to a microarray. The hybridization signal for each gene spotted on the array was determined using a laser confocal scanner. The intensity of the hybridization signal was representative of the expression level for the gene corresponding to that spot. The ratio of CY3 to CY5 signal was indicative of the change in gene expression between the two samples being analyzed.

Statistical Analysis of Microarray Data

The gene chip data was processed by using Affymetrix Microarray Suite (MAS 5.0). The resulting data on the gene expression patterns and expression levels was evaluated to identify differences between control (normal) and CRC tissues; subsequently, differentially expressed gene profiles were compared between the different groups CRCs, based on the mutational status of RPH3AL.

Data analysis included everything from examination of data quality through normalization of intensity to gene selection and clustering. For quality control, array comparability was checked using intensity to examine replicate array variation prior to any data analysis (Chen, D. T 2004a). For the normalization process, normalization methods including lowness smooth function and ANOVA modeling was used for bias correction. Data sets from Affymetrix gene chips were processed by using the Affymetrix Microarray Suite (MAS 5.0). Quality control prior to data analysis was checked using the following criteria: background values less than 100, comparable noise values among the arrays, the percentage of “present call” above 25%, and a 3′ to 5′ ratio less than three. In addition, the 2D image plot was used to assess array comparability (Chen, D. T, 2004a). This approach uses a percentile method to group data, applies a 2D image plot to display the grouped microarray data, and employs an invariant band to quantify degrees of array comparability.

Arrays meeting the above requirement were used for statistical analysis. Normalization based on a lowness smooth function was performed to make array intensity comparable for group comparison. Since the study was interested in identifying the differences in gene expression between control (benign epithelial tissues) and CRC tissues, these normalized values were filtered using the paired t-test for every pair of each tumor location and race category of patients (cutoff, P<0.05). In addition, an ANOVA model was used to examine location and race effects. These procedures were performed using SAM software as this approach provides adjusted p values for false discovery rates to account for simultaneous multiple testing. Additional analyses were implemented to check consistency of the results, including Dchip, RMA, and probe-level rank approaches. Since the gene level analysis ignores probe variation, which may miss altered genes caused by a non-homogeneous probe effect (e.g. alternative splicing genes), the probe rank approach was considered to identify altered genes for the oligonucleotide arrays. The approach applies rank score to normalization, analyzes probe intensity to control for probe effect, and uses a filter with percentage of probe fold change to account for cross-hybridization and alternative splicing (Chen, D. T., A graphical approach for quality control of oligonucleotide array data. J Biopharm Stat, 2004a. 14(3): p. 591-606 and Chen, D. T., S. H. Lin, and S. J. Soong, Gene selection for oligonucleotide array: an approach using PM probe level data. Bioinformatics, 2004b. 20(6): p. 854-62). The selected genes were then examined to find subgroups of genes based on various clustering methods, such as hierarchical clustering, self-organizing maps, and neural networks. Gene classification level analysis was performed using FuncAssociate (http://llama.med.haivard.edu/cgi/func/funcassociate).

TABLE 8

Loss of heterozytosity markers, primers and PCR conditions

PCR Conditions

| LOH | | | Size | Initial | Denatu- | Anneal- | Exten- | | Final |
|---|---|---|---|---|---|---|---|---|---|
| Marker | Types | Primer | (bp) | Denaturation | ration | ing | sion | Cycles | Extension |
| Marker | D17S1866 | S5′TGGATTCTGTAGTCCCAGG 3′ | 154 | 95° C. | 95° C. | 60° C. | 72° C. | 35 | 72° C. |
| | | SEQ ID NO: 23 | | | | | | | |
| | | AS5′GGTTCAAAGACAACTCCCC 3′ | | | | | | | |
| | | SEQ ID NO: 24 | | | | | | | |
| | D17S926 | S5′GCAGTGGGCCATCATCA 3′ | 243 | 95° C. | 95° C. | 60° C. | 72° C. | 35 | 72° C. |
| | | SEQ ID NO: 25 | | | | | | | |
| | | AS5′CCGCAGAAGGCTGTTGT 3′ | | | | | | | |
| | | SEQ ID NO: 26 | | | | | | | |
| | D17S643 | S5′CTTCCTTGTCTCTAAACAGTCCTTT 3′ | 151 | 95° C. | 94° C. | 55° C. | 70° C. | 35 | 70° C. |
| | | SEQ ID NO: 27 | | | | | | | |
| | | AS5′GTAGTCCCAGGGAGCTGGAAGT 3′ | | | | | | | |
| | | SEQ ID NO: 28 | | | | | | | |
| | D17S849 | S5′CAATTCTGTTCTAAGATTATTTTGG 3′ | 254 | 94° C. | 94° C. | 55° C. | 72° C. | 35 | 72° C. |
| | | SEQ ID NO: 29 | | | | | | | |
| | | AS5′CTCTGGCTGAGGAGGC 3′ | | | | | | | |
| | | SEQ ID NO: 30 | | | | | | | |

S = SENSE PRIMER, AS = ANTISENSE PRIMER

TABLE 9
Modulation of RPH3AL expression by LOH based on
the type of genotype of SNP at 5′UTR-25.

| | | | | mRNA mean copy number of RPH3A | | |
|---|---|---|---|---|---|---|
| | LOH status at 1713.3 | | | gene/Beta actin ratio | | |
| | (RPH3AL gene region) | | | | Tumor | |
| Genotype at | | Without | | Tumor | Without | Matching |
| 5′UTR-25 | With LOH | LOH | χ² P-value | with LOH | LOH | Normal |
| A/A | 5/5 | 0/5 | ≧0.001 | 0.38 | none | 2.99 |
| C/A | 1/7 | 6/7 | | 0.16 | 0.60 | 0.78 |
| C/C | 7/10 | 3/10 | | 0.23 | 0.47 | 3.42 |
| Total/Average | 13/22 | 9/22 | | 0.26 | 0.54 | 2.34 |

TABLE 10

Relationship between Single nucleotide polymorphisms within the genomic region of RPH3AL
gene and SNP at 5′UTR-25

RPH3AL genotype at 5′UTR-25

| | | | Position on | A/A (n = 5) | C/A (n = 6) | C/C (n = 10) |
|---|---|---|---|---|---|---|
| SNP | | | chromosome | §No. of | No. of | No. of |
| Cluster ID | Primer sequence (5′-3′) | Alleles | 17 | Mut(%) | Mut(%) | Mut(%) |
| rs498561 | F1: CTTGGAACCCAGTTTGTTGG | G/T | 203345 | 0(0) | 0(0) | 1(10) |
| | SEQ ID NO: 31 | | | | | |
| | R1: ACACCATCAGCAACCTTTCC | | | | | |
| | SEQ ID NO: 32 | | | | | |
| rs7215343 | F1: AGATTTGGGACGCTTCCTCT | C/T | 202893 | 0(0) | 1(17) | 1(10) |
| | SEQ ID NO: 33 | | | | | |
| | R1: TTCTCTGTGCGTCAATTTGC | | | | | |
| | SEQ ID NO: 34 | | | | | |
| rs7223403 | F1: GTCAGCATCTCATCGCTTCC | A/G | 203005 | 0(0) | 1(17) | 1(10) |
| | SEQ ID NO: 35 | | | | | |
| | R1: GTGCAGGGGTTCATTTCATT | | | | | |
| | SEQ ID NO: 36 | | | | | |
| rs9891032 | F1: CCAAGAGGCTCTCAAACTGG | A/G | 203362 | 0(0) | 0(0) | 0(0) |
| | SEQ ID NO: 37 | | | | | |
| | R1: TGGGTTGGGCTGTAATAACC | | | | | |
| | SEQ ID NO: 38 | | | | | |
| rs9907777 | F1: AATTGCCAAGAGGCTCTCAA | C/T | 203367 | 2(40) | 2(33) | 2(20) |
| | SEQ ID NO: 39 | | | | | |
| | R1: CGAGGAAAAAGCTGAGTTGC | | | | | |
| | SEQ ID NO: 40 | | | | | |
| rs9915104 | F1: GAACTACTCGGGAGGCTGAG | C/G | 204137 | 0(0) | 0(0) | 0(0) |
| | SEQ ID NO: 41 | | | | | |
| | R1: CAGGCCTTGGTATGTGTGTG | | | | | |
| | SEQ ID NO: 42 | | | | | |
| rs11356209 | F1: ACACCATCAGCAACCTTTCC | -/A | 203333 | 0(0) | 0(0) | 0(0) |
| | SEQ ID NO: 43 | | | | | |
| | R1: CTTGGAACCCAGTTTGTTGG | | | | | |
| | SEQ ID NO: 44 | | | | | |
| rs11356210 | F1: TCAAACTGGGTTCCAAGGAG | -/A | 203465 | 0(0) | 0(0) | 0(0) |
| | SEQ ID NO: 45 | | | | | |
| | R1: CGAGGAAAAAGCTGAGTTGC | | | | | |
| | SEO ID NO: 46 | | | | | |
| rs11383870 | F1: TTTGGGTTAGAAAGGGCACA | -/A | 202931 | 0(0) | 0(0) | 1(0) |
| | SEQ ID NO: 47 | | | | | |
| | R1: TTCTCTGTGCGTCAATTTGC | | | | | |
| | SEQ ID NO: 48 | | | | | |
| rs11650641 | F1: TAGTGGGGTTTTGAGGGACA | A/G | 61971 | 0(0) | 0(0) | 0(0) |
| | SEQ ID NO: 49 | | | | | |
| | R1: CGGATAAGCAAGGCCATCTA | | | | | |
| | SEQ ID NO: 50 | | | | | |
| rs12942009 | F1: GTGTGGAGACCCTCTTCAGG | C/T | 203456 | 0(0) | 0(0) | 0(0) |
| | SEQ ID NO: 51 | | | | | |
| | R1: CGAGGAAAAAGCTGAGTTGC | | | | | |
| | SEQ ID NO: 52 | | | | | |
| rs12942039 | F1: CAACAAACTGGGTTCCAAGG | C/T | 203498 | 2(40) | 3(50) | 5(50) |
| | SEQ ID NO: 53 | | | | | |
| | R1: TAACCAGCAGCGCATATCAG | | | | | |
| | SEQ ID NO: 54 | | | | | |
| rs12949751 | F1: CAACAAACTGGGTTCCAAGG | A/G | 203500 | 2(40) | 2(33) | 2(20) |
| | SEQ ID NO: 55 | | | | | |
| | R1: TAACCAGGAGCGCATATCAG | | | | | |
| | SEQ ID NO: 56 | | | | | |

§No of mut = Number of mutant genotypes

The data were analyzed using different approaches (Allison, D. B., et al 2002). For each gene, the ordinary and the Empirical Bayes (EB) estimate of the standardized difference was determined. Three different types of p-values were obtained: simple t-test p-values for the original data and log-transformed data both assuming equal variances, and chebby checker p-values. These p-values are presented on an individual basis as well as by taking multiple comparisons into account. The mix-o-matic method is applied to provide additional information about these p-values. The results shown here are the comparisons between control group and the one week castration group. The relationship between the EmpiricalBbayes (EB) and the ordinary estimate (OE) of the standardized difference is shown in FIG. 6. The relationship between the EB and OE estimate appears to be non-monotonic, which is theoretically undesirable. This non-monotonic relationship is possibly caused by the estimated standard error of the OE estimate, which heavily depends on the value of the corresponding OE estimate. Due to this standard error, relatively high values of the OE estimate will shrink the corresponding EB estimate too strongly towards the weighted grand mean over all OE estimates. Aim of this statistical analysis is to find which genes are significantly up regulated or significantly down regulated when AA genotype CRCs were compared with the CC genotype group. This up or down regulation of genes is examined by means of p-values for the null-hypothesis that there is no differential expression between groups and estimates of the standardized difference in these two groups. If for a particular gene this p-value is significant at the 5% level (p-value ≦0.05), then this gene is declared to be up-regulated when the estimated standardized difference is positive and declared to be down-regulated when this estimated is negative. If this p-value is non-significant (p-value >0.05) then this gene is declared to be not influenced. In addition to these initial data processing steps and utilization of additional analyses described above, 29.5% of genes identified were differentially expressed in the AA genotype versus the CC genotype groups.

The quartile approach was used to normalize data. Two-sample test based on SAM approach was used to identify regulated genes. The regulated genes were then classified using gene ontology annotation and KEGG pathway.

Based on the impact of AA allelic forms on the risk of recurrence and survival, it is evident that the AA genotype is associated with aggressive CRC phenotypes. Therefore, the study was focused on the genes involved in the metastatic process. Differentially expressed genes in CRCs with the AA genotype versus the CC genotype group of CRCs are shown in Tables 11 and 12. The genes that are down regulated or up regulated in CRCs with the AA genotype compared to CRCs with the CC genotype demonstrate several key molecules involved in the metastasis.

TABLE 11
Down-Regulated Genes Associated with
Metastasis (AA vs. CC genotype)

| Probe Set | Gene | Fold | | |
|---|---|---|---|---|
| ID | Name | Change | P value | Description |
| 205290_s_at | BMP2 | 2.96 | 0.0082 | bone morphogenetic |
| | | | | protein 2 |
| 208796_s_at | CCNG1 | 1.42 | 0.0036 | cyclin G1 |
| 235639_at | CDH19 | 14.87 | 0.0000 | cadherin 19, type 2 |
| 1555186_at | CDKN1A | 5.78 | 0.0011 | cyclin-dependent |
| | | | | kinase inhibitor |
| | | | | 1A (p21, Cip1) |

TABLE 12
Up-Regulated Genes Associated with Metastasis (AA vs. CC genotype)

| Probe Set | Gene | Fold | | |
|---|---|---|---|---|
| ID | Name | Change | P value | Description |
| 204490_s_at | CD44 | 2.20 | 0.0048 | CD44 antigen (homing function and Indian |
| | | | | blood group system) |
| 209835_x_at | CD44 | 2.14 | 0.0081 | CD44 antigen (homing function and Indian |
| | | | | blood group system) |
| 212014_x_at | CD44 | 2.24 | 0.0030 | CD44 antigen (homing function and Indian |
| | | | | blood group system) |
| 203213_at | CDC2 | 6.56 | 0.0062 | cell division cycle 2, G1 to S and G2 to M |
| 212022_s_at | MKI67 | 3.94 | 0.0026 | antigen identified by monoclonal antibody |
| | | | | Ki-67 |
| 204259_at | MMP7 | 49.55 | 0.0051 | matrix metalloproteinase 7 (matrilysin, |
| | | | | uterine) |
| 201577_at | NME1 | 3.34 | 0.0041 | non-metastatic cells 1, protein (NM23A) |
| | | | | expressed in |
| 200660_at | S100A11 | 3.80 | 0.0002 | S100 calcium binding protein A11 |
| | | | | (calgizzarin) |
| 203625_x_at | SKP2 | 2.89 | 0.0020 | S-phase kinase-associated protein 2 (p45) |
| 201506_at | TGFBI | 3.79 | 0.0040 | transforming growth factor, beta-induced, |
| | | | | 68kDa |
| 201666_at | TIMP1 | 6.68 | 0.0003 | tissue inhibitor of metalloproteinase 1 |
| | | | | (erythroid potentiating activity, collagenase |
| | | | | inhibitor) |
| 211527_x_at | VEGF | 3.65 | 0.0005 | vascular endothelial growth factor |

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. McDonald, J. D., Daneshvar, L., Willert, J. R., Matsumura, K., Waldman, F., and Cogen, P. H. Physical mapping of chromosome 17p13.3 in the region of a putative tumor suppressor gene important in medulloblastoma. Genomics, 23: 229-232, 1994.
2. Biegel, J. A., Burk, C. D., Barr, F. G., and Emanuel, B. S. Evidence for a 17p tumor related locus distinct from p53 in pediatric primitive neuroectodermal tumors. Cancer Res, 52: 3391-3395, 1992.
3. Vogelstein, B., Fearon, E. R., Kern, S. E., Hamilton, S. R., Preisinger, A. C., Nakamura, Y., and White, R. Allelotype of colorectal carcinomas. Science, 244: 207-211, 1989.
4. Vogelstein, B., Fearon, E. R., Hamilton, S. R., Kern, S. E., Preisinger, A. C., Leppert, M., Nakamura, Y., White, R., Smits, A. M., and Bos, J. L. Genetic alterations during colorectal-tumor development. New England Journal of Medicine, 319: 525-532, 1988.
5. Fearon, E. R. and Vogelstein, B. A genetic model for colorectal tumorigenesis. Cell, 61: 759-767, 1990.
6. Thiagalingam, S., Laken, S., Willson, J. K., Markowitz, S. D., Kinzler, K. W., Vogelstein, B., and Lengauer, C. Mechanisms underlying losses of heterozygosity in human colorectal cancers. Proc Natl Acad Sci USA, 98: 2698-2702, 2001.
7. Manne, U., Weiss, H. L., Myers, R. B., Danner, O. K., Moron, C., Srivastava, S., and Grizzle, W. E. Nuclear accumulation of p53 in colorectal adenocarcinoma: prognostic importance differs with race and location of the tumor. Cancer, 83: 2456-2467, 1998.
8. Kern, S. E., Pietenpol, J. A., Thiagalingam, S., Seymour, A., Kinzler, K. W., and Vogelstein, B. Oncogenic forms of p53 inhibit p53-regulated gene expression. Science, 256: 827-830, 1992.
9. Freedman, A. N., Michalek, A. M., Marshall, J. R., Mettlin, C. J., Petrelli, N. J., Black, J. D., Zhang, Z. F., Satchidanand, S., and Asirwatham, J. E. Familial and nutritional risk factors for p53 overexpression in colorectal cancer. Cancer Epidemiology, Biomarkers & Prevention, 5: 285-291, 1996.
10. Delattre, O., Olschwang, S., Law, D. J., Melot, T., Remvikos, Y., Salmon, R. J., Sastre, X., Validire, P., Feinberg, A. P., and Thomas, G. Multiple genetic alterations in distal and proximal colorectal cancer. Lancet, 2: 353-356, 1989.
11. Boland, C. R., Sato, J., Appelman, H. D., Bresalier, R. S., and Feinberg, A. P. Microallelotyping defines the sequence and tempo of allelic losses at tumour suppressor gene loci during colorectal cancer progression. Nature Medicine, 1: 902-909, 1995.
12. Batra, S. K., McLendon, R. E., Koo, J. S., Castelino-Prabhu, S., Fuchs, H. E., Krischer, J. P., Friedman, H. S., Bigner, D. D., and Bigner, S. H. Prognostic implications of chromosome 17p deletions in human medulloblastomas. J Neurooncol, 24: 39-45, 1995.
13. Smith, J. S., Tachibana, I., Allen, C., Chiappa, S. A., Lee, H. K., McIver, B., Jenkins, R. B., and Raffel, C. Cloning of a human ortholog (RPH3AL) of (RNO)Rph3a1 from a candidate 17p13.3 medulloblastoma tumor suppressor locus. Genomics, 59: 97-101, 1999.
14. Kato, M., Sasaki, T., Ohya, T., Nakanishi, H., Nishioka, H., Imamura, M., and Takai, Y. Physical and functional interaction of rabphilin-3A with alpha-actinin. J Biol Chem, 271: 31775-31778, 1996.
15. Kotake, K., Ozaki, N., Mizuta, M., Sekiya, S., Inagaki, N., and Seino, S, Noc2, a putative zinc finger protein involved in exocytosis in endocrine cells. J Biol Chem, 272: 29407-29410, 1997.
16. Goi, T., Takeuchi, K., Katayama, K., Hirose, K., and Yamaguchi, A. Mutations of rabphillin-3A-like gene in colorectal cancers. Oncol Rep, 9: 1189-1192, 2002.
17. Zhang, J., Cui, Y., Kuang, G., Li, Y., Wang, N., Wang, R., Guo, W., Wen, D., Wei, L., Yu, F., and Wang, S. Association of the thymidylate synthase polymorphisms with esophageal squamous cell carcinoma and gastric cardiac adenocarcinoma. Carcinogenesis, 25: 2479-2485, 2004.
18. Nedelcheva Kristensen, V., Haraldsen, E. K., Anderson, K. B., Lonning, P. E., Erikstein, B., Karesen, R., Gabrielsen, O, S., and Borresen-Dale, A. L. CYP17 and breast cancer risk: the polymorphism in the 5′ flanking area of the gene does not influence binding to Sp-1. Cancer Res, 59: 2825-2828, 1999.
19. Hamajima, N., Saito, T., Matsuo, K., and Tajima, K. Competitive amplification and unspecific amplification in polymerase chain reaction with confronting two-pair primers. J Mol Diagn, 4: 103-107, 2002.
20. Fleiss, J. Statistical methods for rates and proportions. New York, N.Y.: John Wiley and Sons, 1981.
21. Kaplan, E. and Meier, P. Non-parametric estimation from incomplete observations. Am Stat Assoc, 53, 1958.
22. Allison, P. Survival Analysis Using the SAS System: A Practical Guide. Cary, N.C.: SAS Institute Inc, 1995.
23. Kleinbaum, D. Survival Analysis: A Self Learning Text. New York, N.Y.: Springer-Verlag, 1996.
24. Park, W. S., Cho, Y. G., Park, J. Y., Kim, C. J., Lee, J. H., Kim, H. S., Lee, J. W., Song, Y. H., Park, C. H., Park, Y. K., Kim, S. Y., Nam, S. W., Lee, S. H., Yoo, N. J., and Lee, J. Y. A single nucleotide polymorphism in the E-cadherin gene promoter-160 is not associated with risk of Korean gastric cancer. J Korean Med Sci, 18: 501-504, 2003.
25. Signori, E., Bagni, C., Papa, S., Primerano, B., Rinaldi, M., Amaldi, F., and Fazio, V. M. A somatic mutation in the 5′UTR of BRCA1 gene in sporadic breast cancer causes down-modulation of translation efficiency. Oncogene, 20: 4596-4600, 2001.
26. Randerson-Moor, J. A., Gaut, R., Turner, F., Whitaker, L., Barrett, J. H., Silva Idos, S., Swerdlow, A., Bishop, D. T., and Bishop, J. A. The relationship between the epidermal growth factor (EGF) 5′UTR variant A61G and melanoma/nevus susceptibility. J Invest Dermatol, 123: 755-759, 2004.
27. Sakano, S., Berggren, P., Kumar, R., Steineck, G., Adolfsson, J., Onelov, E., Hemminki, K., and Larsson, P. Clinical course of bladder neoplasms and single nucleotide polymorphisms in the CDKN2A gene. Int J Cancer, 104: 98-103, 2003.
28. Katkoori, V., Gandham, M., Jhala, N., Soong, R., Diasio, R. B., Meleth, S., Grizzle, W. E., and Manne, U. Differences in the mutational spectra of the p53 gene in proximal colonic adenocarcinomas of African Americans and Caucasians. In: American Association for Cancer Research 2005, pp. Abstract 4023, pp. 4930.
29. Marsh, S., Collie-Duguid, E. S. R., Li, T., Liu, X., and McLeod, H. L. Ethnic variation in the thymidylate synthase enhancer region polymorphism among Caucasian and Asian populations. Genomics, 58: 310-312, 1999.
30. Jin, X., Wu, X., Roth, J. A., Amos, C. I., King, T. M., Branch, C., Honn, S. E., and Spitz, M. R. Higher lung cancer risk for younger African-Americans with the Pro/Pro p53 genotype. Carcinogenesis, 16: 2205-2208, 1995.
31. Katkoori, V., Chatla, C., Jia, X., and Manne, U. Mutations in the L3 loop of zinc-binding domain of p53 are poor prognostic indicators of Caucasians with colorectal adenocarcinomas. In: American Association of Cancer Research 2005, pp. Abstract #2548.
32. Kawakami, K., Ishida, Y., Danenberg, K. D., Omura, K., Watanabe, G., Danenberg, P. V. Functional polymorphism of the thymidylate synthase gene in colorectal cancer accompanied by frequent loss of heterozygosity, Jpn J Cancer Res, 93 (11), 1221-9, 2002,
33. Wieland, I., Arden, K. C., Michels, D., Klein-Hitpass, L., Bohm, M., Viars, C. S., Weidle, U. H. Isolation of DICE1: a gene frequently affected by LOH and downregulated in lung carcinomas, Oncogene, 18 (32), 4530-7, 1999.
34. Shirasaki, F., Takata, M., Hatta, N., Takehara, K. Loss of expression of the metastasis suppressor gene KiSS1 during melanoma progression and its association with LOH of chromosome 6q16.3-q23. Cancer Res, 61 (20), 7422-5, 2001.
35. Liloglou, T., Maloney, P., Xinarianos, G., Fear, S., and Field, J. K. Sensitivity and limitations of high throughput fluorescent microsatellite analysis for the detection of allelic imbalance: application in lung tumors. Int J Oncol, 16: 5-14, 2000.
36. Liloglou, T., Maloney, P., Xinarianos, G., Hulbert, M., Walshaw, M. J., Gosney, J. R., Turnbull, L., and Field, J. K. Cancer-specific genomic instability in bronchial lavage: a molecular tool for lung cancer detection. Cancer Res, 61: 1624-1628, 2001.
37. Allison, D. B., Gadbury, G., Heo, M., Fernandez, J., Lee, C-K., Prolla, T. A., & Weindruch, R., A Mixture Model Approach For The Analysis of Microarray Gene Expression Data. Computational Statistics & Data Analysis, 2002. 39: p. 1-20.
38. Chen, D. T., A graphical approach for quality control of oligonucleotide array data. Biopharm Stat, 2004a. 14(3): p. 591-606.
39. Chen, D. T., S. H. Lin, and S. J. Soong, Gene selection for oligonucleotide array: an approach using PM probe level data. Bioinformatics, 2004b. 20(6): p. 854-62.

What is claimed is:

1. A method for determining or predicting aggressiveness of a subject's tumor, comprising comparing the subject's Rabphillin-3A-Like gene genotype with one or more reference genotypes, wherein the reference genotype or genotypes correlate with aggressive tumor growth, a similar genotype in the subject's Rabphillin-3A-Like gene as compared to the reference genotype or genotypes indicating an aggressive tumor in the subject.

2. The method of claim 1, further comprising comparing other nucleotides within the genomic region of the subject's Rabphillin-3A-Like gene genotype with nucleotides in one or more reference genotypes, wherein the reference genotype or genotypes correlate with aggressive tumor growth, a similar nucleotide within the genomic region of the subject's Rabphillin-3A-Like gene as compared to the reference genotype or genotypes indicating an aggressive tumor in the subject.

3. The method of claim 1 wherein the tumor is malignant.

4. The method of claim 1 wherein the tumor is a colorectal tumor.

5. The method of claim 1, wherein the reference genotype comprises the nucleotides at position −25 of the 5′ untranslated regions of exon 2 of the Rabphillin-3A-Like gene.

6. The method of claim 5, wherein the reference genotype is A/A.

7. A method for determining a subject's predisposition to cancer, comprising comparing the subject's Rabphillin-3A-Like gene genotype with one or more reference genotypes, wherein the reference genotype or genotypes correlate with a predisposition to cancer, a similar genotype in the subject's Rabphillin-3A-Like gene as compared to the reference genotype or genotypes indicating the subject's predisposition to cancer.

8. The method of claim 7, further comprising comparing other nucleotides within the genomic region of the subject's Rabphillin-3A-Like gene genotype with nucleotides in one or more reference genotypes, wherein the reference genotype or genotypes correlate with aggressive tumor growth, a similar nucleotide within the genomic region of the subject's Rabphillin-3A-Like gene as compared to the reference genotype or genotypes indicating the subject's predisposition to cancer.

9. The method of claim 7, wherein the tumor is malignant.

10. The method of claim 7, wherein the tumor is a colorectal tumor.

11. The method of claim 7, wherein the reference genotype comprises the nucleotides at position −25 of the 5′ untranslated regions of exon 2 of the Rabphillin-3A-Like gene.

12. The method of claim 11, wherein the reference genotype is A/A.

13. A method of diagnosing cancer in a subject, comprising, comparing the subject's Rabphillin-3A-Like gene genotype with one or more reference genotypes, wherein the reference genotype or genotypes correlate with cancer, a similar genotype in the subject's Rabphillin-3A-Like gene as compared to the reference genotype or genotypes indicating the subject's cancer.

14. The method of claim 13, further comprising comparing other nucleotides within the genomic region of the subject's Rabphillin-3A-Like gene genotype with nucleotides in one or more reference genotypes, wherein the reference genotype or genotypes correlate with aggressive tumor growth, a similar nucleotide within the genomic region of the subject's Rabphillin-3A-Like gene as compared to the reference genotype or genotypes indicating the subject's cancer.

15. The method of claim 13, wherein the tumor is malignant.

16. The method of claim 13, wherein the tumor is a colorectal tumor.

17. The method of claim 13, wherein the reference genotype comprises the nucleotides at position −25 of the 5′ untranslated regions of exon 2 of the Rabphillin-3A-Like gene.

18. The method of claim 17, wherein the reference genotype is A/A.

19. A method of selecting a therapy for a subject with cancer, comprising:
a. comparing the subject's Rabphillin-3A-Like gene genotype with one or more reference Rabphillin-3A-Like gene genotypes, wherein each reference Rabphillin-3A-Like gene genotype is assigned a preferred therapy; and
b. selecting the reference Rabphillin-3A-Like gene genotype most similar to the subject's Rabphillin-3A-Like gene genotype, the preferred therapy for the most similar reference genotype is the selected therapy for the subject.

20. A kit for determining a Rabphillin-3A-Like gene genotype in a subject, comprising:
a. one or more amplification primers selected from the group consisting of SEQ ID NOS:11-14; and
b. instructions for determining the subject's genotype using one or more of the amplification primers.

21. A computer system for determining matching genotypes between test genotypes and reference genotypes, comprising:
a. a database having a plurality of records, each of said records containing a reference genotype comprising the SNP in the 5′ UTR region of exon 2 of the Rabphillin-3A-Like gene and associated diagnosis, disease prediction, prognosis and therapy data; and
b. a user interface allowing a user to selectively query the database with one or more test genotypes and to display the records associated with matching genotypes.

22. The computer system of claim 22, wherein the SNP in the Rabphillin-3A-Like gene comprises the nucleotides at position −25 of the 5′ untranslated region of the exon 2 of the Rabphillin-3A-Like gene.

23. A method for determining a Rabphillin-3A-Like gene genotype in a subject comprising identifying nucleotides present in both copies in the subject's Rabphillin-3A-Like gene at a polymorphic site in the 5′ untranslated region of exon 2 of the Rabphillin-3A-Like gene.

24. The method of claim 23, wherein the identifying step comprises identifying the nucleotide at position −25 of the 5′ untranslated region of the exon 2 of the Rabphillin-3A-Like gene.

25. The method of claim 24, wherein the genotype is selected from the group consisting of C/C, C/A, and A/A.

26. The method of claim 23, wherein the nucleotides are identified by sequencing or probing.

27. The method of claim 23, further comprising amplifying nucleic acids sequences from the subject's Rabphillin-3A-Like gene.

28. The method of claim 27, wherein the amplifying step comprises a polymerase chain reaction-confronting two pair primer method (PCR-CTPP).

29. The method of claim 28, wherein the primers used in the PCR-CTPP comprises one or more of the nucleic acid sequences selected from the group consisting of

| | | |
|---|---|---|
| GAGGGCACAGAGAACCTGTC | (F1 primer), | (SEQ ID NO: 11) |
| GGAGCACCCGGCTGGGGGTT | (R1 primer), | (SEQ ID NO: 12) |
| CATCTCAGATGTGACTCCCC | (F21 primer), | (SEQ ID NO: 13) |
| and | | |
| GGCCCCAGAGGTACTCACTT | (R2 primer). | (SEQ ID NO: 14) |

30. A method of characterizing a Rabphillin-3A-Like gene in a subject comprising the steps of
a. obtaining a biological sample from the subject, wherein the sample comprises a nucleic acid of human Rabphillin-3A-Like gene;
b. amplifying the nucleic acid with a polymerase chain reaction-confronting two pair primer method (PCR-CTPP); wherein the PCR-CTPP method comprises contacting the nucleic acid with one or more primers comprising the sequence of GAGGGCACAGAGAACCTGTC (F1 primer) (SEQ ID NO:11), GGAGCACCCGGCTGGGGGTT (R1 primer) (SEQ ID NO:12), CATCTCAGATGTGACTCCCC (F21 primer) (SEQ ID NO:13), or GGCCCCAGAGGTACTCACTT (R2 primer) (SEQ ID NO:14); and.
c. identifying nucleotides at a polymorphic site in the 5′ untranslated region of exon 2 of the Rabphillin-3A-Like gene, the identified nucleotide indicating the character of the polymorphic Rabphillin-3A-Like gene.

31. The method of claim 30, wherein the polymorphic site is at position −25 of the 5′ untranslated region of exon 2 of the Rabphillin-3A-Like gene.

32. The method of claim 31, wherein the polymorphic site comprises a C or an A at this position.

33. A computer program product comprising a computer readable medium for determining matching genotypes between test genotypes and reference genotypes, comprising a code means for comparing the subject's Rabphillin-3A-like gene genotype with one or more reference genotypes, wherein the reference genotype or genotypes have associated diagnosis, disease prediction, prognosis and therapy data and code means for displaying results of said comparison.


SEQUENCE LISTING (56 sequences)

SEQ ID NO: 1 - DNA, 2611 bp, Artificial Sequence
  Description of Artificial Sequence; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 2 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactcccaacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 3 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagccctgcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 4 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggccgagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 5 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgttgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 6 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacatcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 7 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgtggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 8 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgactcccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 9 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctgggcagtggtgagacggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 10 - DNA, 2611 bp, Artificial Sequence
  Description of Artificial Sequences; note = synthetic construct
dacaacggtggtgggagggagagcggcctcctcctccctggtggggcctgtctgggtgaagcccctctgttcccgaggatcgtctgccttattgaaaccatctaattggagatccgcagattaggctaccaagacacagctgcctccagcagaatttgtgtagtggacacttgtacgtgcactttggagacagcaagcatgaggcctgcagctgacagctttgagtggctgtgaacctgactatggaaaagatgtgactccccacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgatagtgactcggatcttagctcctccagcctagaggacagactcccatccactggggtcagggaccggaaaggcgacaaaccctggaaggagtcaggtggcagcgtggaggcccccaggatggggttcacccacccgccgggccacctctctgggtgccagagcagcctggccagtggtgagacggggacaggctctgctgacccgccagggggaccccgccccgggctgacccgaaaggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccgtgggctggcccaggaccgcaggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgttcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgttcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaaaaaaaaaaaaaaaaaaaaaaaaaaaa

SEQ ID NO: 11 - DNA, 21 bp, Artificial Sequence
  Description of Artificial Sequences; note = synthetic construct
dgagggcacagagaacctgtc

SEQ ID NO: 12 - DNA, 21 bp, Artificial Sequence
  Description of Artificial Sequences; note = synthetic construct
dggagcacccggctgggggtt

SEQ ID NO: 13 - DNA, 21 bp, Artificial Sequence
  Description of Artificial Sequences; note = synthetic construct
dcatctcagatgtgactcccc

SEQ ID NO: 14 - DNA, 21 bp, Artificial Sequence
  Description of Artificial Sequences; note = synthetic construct
dggccccagaggtactcactt

SEQ ID NO: 15 - DNA, 21 bp, Artificial Sequence
  Description of Artificial Sequences; note = synthetic construct
dgtgcactttggagacagcaa

SEQ ID NO: 16 - DNA, 21 bp, Artificial Sequence
  Description of Artificial Sequences; note = synthetic construct
dgtgggaggggagggtaataa

SEQ ID NO: 17 - DNA, 19 bp, Artificial Sequence
  Description of Artificial Sequences; note = synthetic construct
dgctttccacgacggtgac

SEQ ID NO: 18 - DNA, 21 bp, Artificial Sequence
  Description of Artificial Sequences; note = synthetic construct
acccaaaacccaaaatggcag

SEQ ID NO: 19 - DNA, 21 bp, Artificial Sequence
  Description of Artificial Sequences; note = synthetic construct
dcgaggatcgtctgccttatt

SEQ ID NO: 20 - DNA, 22 bp, Artificial Sequence
  Description of Artificial Sequences; note = synthetic construct
gcacgtacaagtgtccactaca

SEQ ID NO: 21 - DNA, 25 bp, Artificial Sequence
  Description of Artificial Sequences; note = synthetic construct
dtaagtaggcgcacagtaggtctga

SEQ ID NO: 22 - DNA, 22 bp, Artificial Sequence
  Description of Artificial Sequences; note = synthetic construct
aagtgcaaagaacacggctaag

SEQ ID NO: 23 - DNA, 20 bp, Artificial Sequence
  Description of Artificial Sequences; note = synthetic construct
dtggattctgtagtcccagg

SEQ ID NO: 24 - DNA, 20 bp, Artificial Sequence
  Description of Artificial Sequences; note = synthetic construct
dggttcaaagacaactcccc

SEQ ID NO: 25 - DNA, 18 bp, Artificial Sequence
  Description of Artificial Sequences; note = synthetic construct
dgcagtgggccatcatca

SEQ ID NO: 26 - DNA, 18 bp, Artificial Sequence
  Description of Artificial Sequences; note = synthetic construct
dccgcagaaggctgttgt

SEQ ID NO: 27 - DNA, 26 bp, Artificial Sequence
  Description of Artificial Sequences; note = synthetic construct
dcttccttgtctctaaacagtccttt

SEQ ID NO: 28 - DNA, 23 bp, Artificial Sequence
  Description of Artificial Sequences; note = synthetic construct
dgtagtcccagggagctggaagt

SEQ ID NO: 29 - DNA, 26 bp, Artificial Sequence
  Description of Artificial Sequences; note = synthetic construct
dcaattctgttctaagattattttgg

SEQ ID NO: 30 - DNA, 17 bp, Artificial Sequence
  Description of Artificial Sequences; note = synthetic construct
dctctggctgaggaggc

SEQ ID NO: 31 - DNA, 22 bp, Artificial Sequence
  Description of Artificial Sequences; note = synthetic construct
ddcttggaacccagtttgttgg

SEQ ID NO: 32 - DNA, 21 bp, Artificial Sequence
  Description of Artificial Sequences; note = synthetic construct
dacaccatcagcaacctttcc

SEQ ID NO: 33 - DNA, 21 bp, Artificial Sequence
  Description of Artificial Sequences; note = synthetic construct
dagatttgggacgcttcctct

SEQ ID NO: 34 - DNA, 21 bp, Artificial Sequence
  Description of Artificial Sequences; note = synthetic construct
dttctctgtgcgtcaatttgc

SEQ ID NO: 35 - DNA, 21 bp, Artificial Sequence
  Description of Artificial Sequences; note = synthetic construct
dgtcagcatctcatcgcttcc

SEQ ID NO: 36 - DNA, 21 bp, Artificial Sequence
  Description of Artificial Sequences; note = synthetic construct
dgtgcaggggttcatttcatt

SEQ ID NO: 37 - DNA, 21 bp, Artificial Sequence
  Description of Artificial Sequences; note = synthetic construct
dccaagaggctctcaaactgg

SEQ ID NO: 38 - DNA, 21 bp, Artificial Sequence
  Description of Artificial Sequences; note = synthetic construct
dtgggttgggctgtaataacc

SEQ ID NO: 39 - DNA, 21 bp, Artificial Sequence
  Description of Artificial Sequences; note = synthetic construct
daattgccaagaggctctcaa

SEQ ID NO: 40 - DNA, 21 bp, Artificial Sequence
  Description of Artificial Sequences; note = synthetic construct
dcgaggaaaaagctgagttgc

SEQ ID NO: 41 - DNA, 21 bp, Artificial Sequence
  Description of Artificial Sequences; note = synthetic construct
dcaactactcgggaggctgag

SEQ ID NO: 42 - DNA, 21 bp, Artificial Sequence
  Description of Artificial Sequences; note = synthetic construct
dcaggccttggtatgtgtgtg

SEQ ID NO: 43 - DNA, 21 bp, Artificial Sequence
  Description of Artificial Sequences; note = synthetic construct
dacaccatcagcaacctttcc

SEQ ID NO: 44 - DNA, 21 bp, Artificial Sequence
  Description of Artificial Sequences; note = synthetic construct
dcttggaacccagtttgttgg

SEQ ID NO: 45 - DNA, 21 bp, Artificial Sequence
  Description of Artificial Sequences; note = synthetic construct
dtcaaactgggttccaaggag

SEQ ID NO: 46 - DNA, 21 bp, Artificial Sequence
  Description of Artificial Sequences; note = synthetic construct
dcgaggaaaaagctgagttgc

SEQ ID NO: 47 - DNA, 21 bp, Artificial Sequence
  Description of Artificial Sequences; note = synthetic construct
dtttgggttagaaagggcaca

SEQ ID NO: 48 - DNA, 21 bp, Artificial Sequence
  Description of Artificial Sequences; note = synthetic construct
dttctctgtgcgtcaatttgc

SEQ ID NO: 49 - DNA, 21 bp, Artificial Sequence
  Description of Artificial Sequences; note = synthetic construct
dtagtggggttttgagggaca

SEQ ID NO: 50 - DNA, 21 bp, Artificial Sequence
  Description of Artificial Sequences; note = synthetic construct
dcggataagcaaggccatcta

SEQ ID NO: 51 - DNA, 21 bp, Artificial Sequence
  Description of Artificial Sequences; note = synthetic construct
dgtgtggagaccctcttcagg

SEQ ID NO: 52 - DNA, 21 bp, Artificial Sequence
  Description of Artificial Sequences; note = synthetic construct
dcgaggaaaaagctgagttgc

SEQ ID NO: 53 - DNA, 21 bp, Artificial Sequence
  Description of Artificial Sequences; note = synthetic construct
dcaacaaactgggttccaagg

SEQ ID NO: 54 - DNA, 21 bp, Artificial Sequence
  Description of Artificial Sequences; note = synthetic construct
dtaaccagcagcgcatatcag

SEQ ID NO: 55 - DNA, 21 bp, Artificial Sequence
  Description of Artificial Sequences; note = synthetic construct
dcaacaaactgggttccaagg

SEQ ID NO: 56 - DNA, 21 bp, Artificial Sequence
  Description of Artificial Sequences; note = synthetic construct
dtaaccagcagcgcatatcag